SEQUENCE LISTING (95 sequences)

SEQ ID NO: 1 - DNA, 1900 bp, Homo sapiens
cgccccacccccactcccgccccggccgtgccctgccggcagggccccccacccccaccccgggccgcgggctcggcctgccccttacgacggcgcccgggccaggaatgttgcatgaatcgtcctgtttgctgttgctcggagactcgccctgtacattgcttagtgccctcaccggccgcccagcccacccagcgcacagtcaggaagggcgtggaccagggaggctggggcgggaggtgccgggggtggggtgccctagcgtgaccacctccttcgcagctcctggtggccattctcccagagggggaacctagtccagcatgcgaggtcaggacccgccttggtgactcggggggaggggggagacattgggattctcgatgggggccaaggagcccccctgttttgcatattttaatccactctatatttggaacgagaaaaggaacaaatatctctgtccgtaatagtttcctctcccctcccttctacttccactggtcccactgcagctgcccagtcttccatctccggcccctcactgccactgccaccccacaacggggcaggggacgctccagctggtctggggttggccagggccctagtggcccgccctggggccccagctcggcccctcgcctcgctgagctctagtgtgccccaccgacccttcaggtgctgctcgtggtgggaggggcggcaggccgcgggtcctgctgtgcacccgcgggaccagccggcctgggagaccatcggccgggggggatgagggcagggccctgccgctccaccgcagccatcttcctcacagggtctctccccaaggagggggctagcttggtccccatgctcttgggcaactacagcagagaagcctccctgccttggaccccaaagtctcctgtcctgccctttatgtgtgtgggtgaaactgggtgcgtctgagcacgtgggagccgtgtgtgtgcctgattactgagtggccaccaggggccgctctggactagcgcggggccgtggaggcgtgcaccgtgtgcatgcgtggggtgtacctgtgagagcaccctgtctcctcttccaaagaaagtcagaggccatcctgcaccctgggtccagctgtttgcccagcctgtccttccagagcctcacccagcctgagcggggttccctggtgaatccctgctgcttggggaggccccaagggccccttggaggcagcgcccccaccttgggcttctgagggcatcatagggggacccctagagtcagttcaccacaggccctggggagagtcaaagacccccgagggtgcccagccccccacactgtgactcctcacactcagcgatgacctgtggggtggggggccctgggacgtttttaaacctagggtttggagtctggactaagctccatccacgtcactcacaagtttctgtttatatttctagctttttttaataaaataaaaaaaaaaagaaaacagaagttttcacaacccaggggcctggcacgccggtctgtgcctgcccgccccgccctggcccaccggccccactccctgggcacagagtcacacccactcatccttccgccaacagtccaggtcacacagcagcagtcactgtaacagactgccacatacacactcggtctcacactcacctgtgggttttggttccgttcaatttgggtttttaactttacagggtcagttccgcttcacctccttttgtatggagttccatccggggggtttcaccccctgctccagtcctgaggcctcctgaccctgacgttgtgatacgccccacagagatctatgtttcttatattattattattgataataattattataatattattatgtaataaatttataagaaatgaa

SEQ ID NO: 2 - DNA, 600 bp, Artificial Sequence
  Reverse complement of bp 1 to 600 of SEQ ID NO1
gccaaccccagaccagctggagcgtcccctgccccgttgtggggtggcagtggcagtgaggggccggagatggaagactgggcagctgcagtgggaccagtggaagtagaagggaggggagaggaaactattacggacagagatatttgttccttttctcgttccaaatatagagtggattaaaatatgcaaaacaggggggctccttggcccccatcgagaatcccaatgtctcccccctccccccgagtcaccaaggcgggtcctgacctcgcatgctggactaggttccccctctgggagaatggccaccaggagctgcgaaggaggtggtcacgctagggcaccccacccccggcacctcccgccccagcctccctggtccacgcccttcctgactgtgcgctgggtgggctgggcggccggtgagggcactaagcaatgtacagggcgagtctccgagcaacagcaaacaggacgattcatgcaacattcctggcccgggcgccgtcgtaaggggcaggccgagcccgcggcccggggtgggggtggggggccctgccggcagggcacggccggggcgggagtgggggtggggcg

SEQ ID NO: 3 - DNA, 201 bp, Artificial Sequence
  Reverse complement of bp 100 to 300 of SEQ ID NO1
gagaatggccaccaggagctgcgaaggaggtggtcacgctagggcaccccacccccggcacctcccgccccagcctccctggtccacgcccttcctgactgtgcgctgggtgggctgggcggccggtgagggcactaagcaatgtacagggcgagtctccgagcaacagcaaacaggacgattcatgcaacattcctggcc

SEQ ID NO: 4 - DNA, 50 bp, Artificial Sequence
  AON2
tggcccgggcgccgtcgtaaggggcaggccgagcccgcggcccggggtgg

SEQ ID NO: 5 - DNA, 50 bp, Artificial Sequence
  AON3
cagggcgagtctccgagcaacagcaaacaggacgattcatgcaacattcc

SEQ ID NO: 6 - DNA, 50 bp, Artificial Sequence
  AON4
gactgtgcgctgggtgggctgggcggccggtgagggcactaagcaatgta

SEQ ID NO: 7 - DNA, 50 bp, Artificial Sequence
  AON5
ccccacccccggcacctcccgccccagcctccctggtccacgcccttcct

SEQ ID NO: 8 - DNA, 50 bp, Artificial Sequence
  AON9
ttgttccttttctcgttccaaatatagagtggattaaaatatgcaaaaca

SEQ ID NO: 9 - DNA, 50 bp, Artificial Sequence
  AON10
ccagtggaagtagaagggaggggagaggaaactattacggacagagatat

SEQ ID NO: 10 - DNA, 50 bp, Artificial Sequence
  AON11
gcagtggcagtgaggggccggagatggaagactgggcagctgcagtggga

SEQ ID NO: 11 - DNA, 50 bp, Artificial Sequence
  AON12
cctggccaaccccagaccagctggagcgtcccctgccccgttgtggggtg

SEQ ID NO: 12 - DNA, 18 bp, Artificial Sequence
  Synthesized
tccacgcccttcctgact

SEQ ID NO: 13 - DNA, 18 bp, Artificial Sequence
  Synthesized
aagcaatgtacagggcga

SEQ ID NO: 14 - DNA, 18 bp, Artificial Sequence
  Synthesized
ggcacctcccgccccagc

SEQ ID NO: 15 - DNA, 18 bp, Artificial Sequence
  Synthesized
tcctgactgtgcgctggg

SEQ ID NO: 16 - DNA, 18 bp, Artificial Sequence
  Synthesized
tgggctgggcggccggtg

SEQ ID NO: 17 - DNA, 18 bp, Artificial Sequence
  Synthesized
agggcactaagcaatgta

SEQ ID NO: 18 - DNA, 50 bp, Artificial Sequence
  scr (scrambled)
ggcgctccgatggttccaatgaacacgtcgcccacgccggatgagcttct

SEQ ID NO: 19 - DNA, 50 bp, Artificial Sequence
  AON1
gggtggggggccctgccggcagggcacggccggggcgggagtgggggtgg

SEQ ID NO: 20 - protein, 50 aa, Artificial Sequence
  AON6
CTGGGAGAATGGCCACCAGGAGCTGCGAAGGAGGTGGTCACGCTAGGGCA

SEQ ID NO: 21 - DNA, 50 bp, Artificial Sequence
  AON7
cgagtcaccaaggcgggtcctgacctcgcatgctggactaggttccccct

SEQ ID NO: 22 - DNA, 50 bp, Artificial Sequence
  AON8
ggggggctccttggcccccatcgagaatcccaatgtctcccccctccccc

SEQ ID NO: 23 - DNA, 50 bp, Artificial Sequence
  AON13
ctcagcgaggcgaggggccgagctggggccccagggcgggccactagggc

SEQ ID NO: 24 - DNA, 50 bp, Artificial Sequence
  AON14
gcccctcccaccacgagcagcacctgaagggtcggtggggcacactagag

SEQ ID NO: 25 - DNA, 50 bp, Artificial Sequence
  AON15
ctcccaggccggctggtcccgcgggtgcacagcaggacccgcggcctgcc

SEQ ID NO: 26 - DNA, 50 bp, Artificial Sequence
  AON16
ggctgcggtggagcggcagggccctgccctcatcccccccggccgatggt

SEQ ID NO: 27 - DNA, 50 bp, Artificial Sequence
  AON17
atggggaccaagctagccccctccttggggagagaccctgtgaggaagat

SEQ ID NO: 28 - DNA, 50 bp, Artificial Sequence
  AON18
actttggggtccaaggctgggaggcttctctgctgtcgttgcccaagagc

SEQ ID NO: 29 - DNA, 50 bp, Artificial Sequence
  AON19
tgctcagacgcacccagtttcacccacacacataaagggcaggacaggag

SEQ ID NO: 30 - DNA, 50 bp, Artificial Sequence
  AON20
agcggcccctggtggccactcagtaatcaggcacacacacggctcccacg

SEQ ID NO: 31 - DNA, 50 bp, Artificial Sequence
  AON21
accccacgcatgcacacggtgcacgcctccacggccccgcgctagtccag

SEQ ID NO: 32 - DNA, 50 bp, Artificial Sequence
  AON22
atggcctctgactttctttggaagaggagacagggtgctctcacaggtac

SEQ ID NO: 33 - DNA, 50 bp, Artificial Sequence
  AON23
tgaggctctggaaggacaggctgggcaaacagctggacccagggtgcagg

SEQ ID NO: 34 - DNA, 50 bp, Artificial Sequence
  AON24
gggcctccccaagcagcagggattcaccagggaaccccgctcaggctggg

SEQ ID NO: 35 - DNA, 50 bp, Artificial Sequence
  AON25
tgatgccctcagaagcccaaggtgggggcgctgcctccaaggggcccttg

SEQ ID NO: 36 - DNA, 50 bp, Artificial Sequence
  AON26
ctttgactctccccagggcctgtggtgaactgactctaggggtcccccta

SEQ ID NO: 37 - DNA, 50 bp, Artificial Sequence
  AON27
tcgctgagtgtgaggagtcacagtgtggggggctgggcaccctcgggggt

SEQ ID NO: 38 - DNA, 50 bp, Artificial Sequence
  AON28
tccaaaccctaggtttaaaaacgtcccagggccccccaccccacaggtca

SEQ ID NO: 39 - DNA, 50 bp, Artificial Sequence
  AON29
gaaatataaacagaaacttgtgagtgacgtggatggagcttagtccagac

SEQ ID NO: 40 - DNA, 50 bp, Artificial Sequence
  AON30
ttgtgaaaacttctgttttctttttttttttattttattaaaaaaagcta

SEQ ID NO: 41 - DNA, 50 bp, Artificial Sequence
  AON31
tgggccagggcggggcgggcaggcacagaccggcgtgccaggcccctggg

SEQ ID NO: 42 - DNA, 50 bp, Artificial Sequence
  AON32
ttggcggaaggatgagtgggtgtgactctgtgcccagggagtggggccgg

SEQ ID NO: 43 - DNA, 50 bp, Artificial Sequence
  AON33
tgtgtatgtggcagtctgttacagtgactgctgctgtgtgacctggactg

SEQ ID NO: 44 - DNA, 50 bp, Artificial Sequence
  AON34
aaaacccaaattgaacggaaccaaaacccacaggtgagtgtgagaccgag

SEQ ID NO: 45 - DNA, 50 bp, Artificial Sequence
  AON35
atggaactccatacaaaaggaggtgaagcggaactgaccctgtaaagtta

SEQ ID NO: 46 - DNA, 50 bp, Artificial Sequence
  AON36
cgtcagggtcaggaggcctcaggactggagcagggggtgaaaccccccgg

SEQ ID NO: 47 - DNA, 50 bp, Artificial Sequence
  AON37
atcaataataataatataagaaacatagatctctgtggggcgtatcacaa

SEQ ID NO: 48 - DNA, 18 bp, Artificial Sequence
  Synthesized
ccccacccccggcacctc

SEQ ID NO: 49 - DNA, 18 bp, Artificial Sequence
  Synthesized
ccgccccagcctccctgg

SEQ ID NO: 50 - DNA, 18 bp, Artificial Sequence
  Synthesized
gtgcgctgggtgggctgg

SEQ ID NO: 51 - DNA, 18 bp, Artificial Sequence
  Synthesized
gcggccggtgagggcact

SEQ ID NO: 52 - DNA, 18 bp, Artificial Sequence
  Synthesized
ctagggcaccccaccccc

SEQ ID NO: 53 - DNA, 18 bp, Artificial Sequence
  Synthesized
ctccctggtccacgccct

SEQ ID NO: 54 - DNA, 18 bp, Artificial Sequence
  Synthesized
agggcaccccacccccgg

SEQ ID NO: 55 - DNA, 18 bp, Artificial Sequence
  Synthesized
cacctcccgccccagcct

SEQ ID NO: 56 - DNA, 18 bp, Artificial Sequence
  Synthesized
ccctggtccacgcccttc

SEQ ID NO: 57 - DNA, 18 bp, Artificial Sequence
  Synthesized
ctgactgtgcgctgggtg

SEQ ID NO: 58 - DNA, 18 bp, Artificial Sequence
  Synthesized
ggctgggcggccggtgag

SEQ ID NO: 59 - DNA, 18 bp, Artificial Sequence
  Synthesized
ggcactaagcaatgtaca

SEQ ID NO: 60 - DNA, 18 bp, Artificial Sequence
  Synthesized
ggcaccccacccccggca

SEQ ID NO: 61 - DNA, 18 bp, Artificial Sequence
  Synthesized
cctcccgccccagcctcc

SEQ ID NO: 62 - DNA, 18 bp, Artificial Sequence
  Synthesized
ctggtccacgcccttcct

SEQ ID NO: 63 - DNA, 18 bp, Artificial Sequence
  Synthesized
gactgtgcgctgggtggg

SEQ ID NO: 64 - DNA, 18 bp, Artificial Sequence
  Synthesized
ctgggcggccggtgaggg

SEQ ID NO: 65 - DNA, 18 bp, Artificial Sequence
  Synthesized
cactaagcaatgtacagg

SEQ ID NO: 66 - DNA, 18 bp, Artificial Sequence
  Synthesized
caccccacccccggcacc

SEQ ID NO: 67 - DNA, 18 bp, Artificial Sequence
  Synthesized
tcccgccccagcctccct

SEQ ID NO: 68 - DNA, 18 bp, Artificial Sequence
  Synthesized
ggtccacgcccttcctga

SEQ ID NO: 69 - DNA, 18 bp, Artificial Sequence
  Synthesized
ctgtgcgctgggtgggct

SEQ ID NO: 70 - DNA, 18 bp, Artificial Sequence
  Synthesized
gggcggccggtgagggca

SEQ ID NO: 71 - DNA, 18 bp, Artificial Sequence
  Synthesized
ctaagcaatgtacagggc

SEQ ID NO: 72 - DNA, 18 bp, Artificial Sequence
  Synthesized
ccacccccggcacctccc

SEQ ID NO: 73 - DNA, 18 bp, Artificial Sequence
  Synthesized
gccccagcctccctggtc

SEQ ID NO: 74 - DNA, 18 bp, Artificial Sequence
  Synthesized
cacgcccttcctgactgt

SEQ ID NO: 75 - DNA, 18 bp, Artificial Sequence
  Synthesized
gcgctgggtgggctgggc

SEQ ID NO: 76 - DNA, 18 bp, Artificial Sequence
  Synthesized
ggccggtgagggcactaa

SEQ ID NO: 77 - DNA, 18 bp, Artificial Sequence
  Synthesized
gcaatgtacagggcgagt

SEQ ID NO: 78 - DNA, 18 bp, Artificial Sequence
  Synthesized
acccccggcacctcccgc

SEQ ID NO: 79 - DNA, 18 bp, Artificial Sequence
  Synthesized
cccagcctccctggtcca

SEQ ID NO: 80 - DNA, 18 bp, Artificial Sequence
  Synthesized
cgcccttcctgactgtgc

SEQ ID NO: 81 - DNA, 18 bp, Artificial Sequence
  Synthesized
gctgggtgggctgggcgg

SEQ ID NO: 82 - DNA, 18 bp, Artificial Sequence
  Synthesized
ccggtgagggcactaagc

SEQ ID NO: 83 - DNA, 18 bp, Artificial Sequence
  Synthesized
aatgtacagggcgagtct

SEQ ID NO: 84 - DNA, 18 bp, Artificial Sequence
  Synthesized
ccccggcacctcccgccc

SEQ ID NO: 85 - DNA, 18 bp, Artificial Sequence
  Synthesized
cagcctccctggtccacg

SEQ ID NO: 86 - DNA, 18 bp, Artificial Sequence
  Synthesized
cccttcctgactgtgcgc

SEQ ID NO: 87 - DNA, 18 bp, Artificial Sequence
  Synthesized
tgggtgggctgggcggcc

SEQ ID NO: 88 - DNA, 18 bp, Artificial Sequence
  Synthesized
ggtgagggcactaagcaa

SEQ ID NO: 89 - DNA, 18 bp, Artificial Sequence
  Synthesized
tgtacagggcgagtctcc

SEQ ID NO: 90 - DNA, 18 bp, Artificial Sequence
  Synthesized
ccggcacctcccgcccca

SEQ ID NO: 91 - DNA, 18 bp, Artificial Sequence
  Synthesized
gcctccctggtccacgcc

SEQ ID NO: 92 - DNA, 18 bp, Artificial Sequence
  Synthesized
cttcctgactgtgcgctg

SEQ ID NO: 93 - DNA, 18 bp, Artificial Sequence
  Synthesized
ggtgggctgggcggccgg

SEQ ID NO: 94 - DNA, 18 bp, Artificial Sequence
  Synthesized
tgagggcactaagcaatg

SEQ ID NO: 95 - DNA, 18 bp, Artificial Sequence
  Synthesized
tacagggcgagtctccga

(19) United States
(12) Patent Application Publication
(10) Pub. No.: US 2024/0294905 A1
(43) Pub. Date: Sep. 5, 2024

(54) ANTISENSE OLIGONUCLEOTIDES FOR INCREASING SHANK3 EXPRESSION

(71) Applicant: Universität Ulm, Ulm (DE)

(72) Inventors: Tobias Böckers, Laupheim (DE); Stefanie Pfänder, Ulm-Wiblingen (DE); Nadine Stirmlinger, Ulm (DE)

(21) Appl. No.: 17/921,922

(22) PCT Filed: Apr. 26, 2021
(86) PCT No.: PCT/EP2021/060848

§ 371 Date: Oct. 27, 2022

(30) Foreign Application Priority Data
Apr. 27, 2020 (DE) 20171521.6

(51) Int. Cl. C12N 15/113; G01N 33/53

(52) U.S. Cl. CPC C12N 15/113; G01N 33/53; C12N 2310/11; G01N 2800/52

(57) ABSTRACT

The present invention relates to an oligonucleotide specifically binding to a polynucleotide comprising the nucleic acid sequence of SEQ ID NO:1. The present invention also relates to said oligonucleotide for use in medicine and for use in treatment and/or prevention of Shank3 deficiency, preferably for use in treatment and/or prevention of Phelan-McDermid syndrome. The present invention also relates to expression constructs, host cells, and methods related thereto.

### CROSS-REFERENCE TO RELATED APPLICATIONS

This application is a U.S. National Phase of International PCT Application No. PCT/EP2021/060848 filed on Apr. 26, 2021, which claims priority to European Patent Application No. 20171521.6 filed on Apr. 27, 2020, the contents of each application are incorporated herein by reference in their entireties.

### SEQUENCE LISTING

This application incorporates by reference the material in the ASCII text file titled ASD05-56_Revised_Sequence_Listing.txt, which was created on Apr. 19, 2023 and is 21 KB. This application also includes a PDF file that is identical to the ASCII text file titled ASD05-56_Revised_Sequence_Listing.txt.

The present invention relates to an oligonucleotide specifically binding to a polynucleotide comprising the nucleic acid sequence of SEQ ID NO:1. The present invention also relates to said oligonucleotide for use in medicine and for use in treatment and/or prevention of Shank3 deficiency, preferably for use in treatment and/or prevention of Phelan-McDermid syndrome. The present invention also relates to expression constructs, host cells, and methods related thereto.

Reduced expression of the Shank 3 gene is associated with disease, including autism (Wang et al. (2019), Mol Psychiatry; doi.org/10.1038/s41380-018-0324-x) and Phelan-McDermid syndrome (PMDS). PMDS is a syndrome caused by a deletion or mutation on chromosome 22, causing only one functional copy of the Shank3 gene to be present in affected patients. The symptoms associated with PMDS are in particular global developmental delay, intellectual disability, and speech, motor, and gait abnormalities (Phelan and McDermott, 2011). Notably, the Shank3 gene product was found to stabilize synapses (Roussignol et al. (2005), J Neurosci 25(14):3560).

Available therapies for PMDS at present exclusively are symptomatic, including administration of insulin and Lithium, risperidone, or neurotrophic factors like IGF-I, leading to an improvement of single symptoms (e.g. Darville et al., 2016). It could be shown in vitro as well as in mouse models that an increase of Shank3 leads to an improvement of molecular properties of affected cells and of behaviour (Mei et al.; 2016). The Shank3 gene was found to be regulated by several miRNAs, which decrease expression of the gene (Choi et al., 2015).

Antisense oligonucleotides have been used, at least experimentally, in treatment of a variety of diseases, in particular neurodegenerative diseases. For example, onset of Spino-cerebellar Ataxia Type 2 could be delayed in a mouse model by administration of an antisense oligonucleotide (Scoles et al., 2017). Similarly, an antisense oligonucleotide binding to a sequence 3′ of the translation start decreases translation of alpha-4 integrin mRNA, inhibiting lymphocyte and monocyte migration in multiple sclerosis (Evers et al., 2015). In addition, antisense oligonucleotides were discussed for treatment of e.g. Usher syndrome type IIa, USH2A-associated non-syndromic retina degeneration, and Morbus Pompe (U.S. Pat. No. 10,131,910, WO 2015/190922 A1, DE 1998 122954, WO 2003/025144 A2). WO 2017/106382 A1 discloses the use of antisense oligonucleotides to enhance production of correctly spliced mRNAs expressed from genes relevant for central nervous system diseases, including Shank3.

Nonetheless, there is still a need in the art for means and methods improving treatment of Shank3 deficiency diseases.

This problem is addressed by the oligonucleotides, expression constructs, host cells, and methods with the features of the independent claims. Advantageous embodiments which might be realized in an isolated fashion or in any arbitrary combinations are listed in the dependent claims.

In accordance, the present invention relates to an oligonucleotide specifically binding to a polynucleotide comprising the nucleic acid sequence of SEQ ID NO:1.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. Likewise, the term “determining an X” refers to determining one X, as well as as to determining more than one of X, e.g. two, three, or four of X. Also, the term “plurality” relates to a multitude, in an embodiment at least two, in a further embodiment, in a further embodiment at least three in a further embodiment at least for, in a further embodiment at least five, of the indicated items.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value±20%, more preferably +10%, most preferably +5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s). In the context of nucleic acid sequences, the term “essentially identical” indicates a percent identity value of at least 80%, preferably at least 90%, more preferably at least 98%, most preferably at least 99%. As will be understood, the term essentially identical includes 100% identity. The aforesaid applies to the term “essentially complementary” mutatis mutandis. Unless otherwise noted, amino acid and nucleotide symbols are those of WIPO standard ST.25.

The term “polynucleotide”, as used herein, relates to a polynucleotide comprising a nucleic acid sequence as shown in SEQ ID NO: 1, a fragment and/or a variant thereof as specified herein below. Preferably, the polynucleotide comprises at least 10, more preferably at least 12, even more preferably at least 14 continuous bases of nucleotides 100 to 300 of SEQ ID NO:1, preferably of nucleotides 50 to 600 of SEQ ID NO: 1, more preferably of nucleotides 1 to 1000 of SEQ ID NO:1, most preferably comprises the nucleotide sequence of SEQ ID NO:1. Preferably, the polynucleotide is a nucleic acid molecule comprising, preferably consisting of, the nucleic acid sequence of Genbank Acc. No. NM_0013720044.1, i.e. is a gene or RNA encoding a Shank3 polypeptide, preferably is an mRNA encoding a Shank 3 polypeptide. Preferably, the encoded Shank3 polypeptide is a human Shank3 polypeptide, more preferably with Genbank Acc. No. NP_001358973.1. As the skilled person understands, there are several isoforms of the Shank3 protein and, accordingly, the encoding mRNAs, all of which are preferably included as polynucleotides as specified herein. It is to be understood that a polypeptide having an amino acid sequence as detailed above may also be encoded due to the degenerated genetic code by more than one species of polynucleotide. Moreover, the term polynucleotide encompasses variants of the aforementioned specific polynucleotides. Said variants may represent orthologs, paralogs or other homologs of the polynucleotide as specified. The polynucleotide variants, preferably, comprise a nucleic acid sequence characterized in that the sequence can be derived from the aforementioned specific nucleic acid sequences by at least one nucleotide substitution, addition and/or deletion, preferably provided that the polynucleotide is comprised in a gene encoding a Shank3 polypeptide or an RNA transcribed therefrom, preferably an mRNA. Variants also encompass polynucleotides comprising a nucleic acid sequence which is capable of hybridizing to the aforementioned specific nucleic acid sequences, preferably, under stringent hybridization conditions. These stringent conditions are known to the skilled worker and can be found in standard text books. A preferred example for stringent hybridization conditions are hybridization conditions in 6× sodium chloride/sodium citrate (=SSC) at approximately 45° C., followed by one or more wash steps in 0.2×SSC, 0.1% SDS at 50° C. to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks. Alternatively, polynucleotide variants are obtainable by PCR-based techniques such as mixed oligonucleotide primer-based amplification of DNA, i.e. using degenerated primers against conserved domains of the polypeptides of the present invention. Conserved domains of the polypeptides of the present invention may be identified by a sequence comparison of the nucleic acid sequence of the polynucleotide or of the amino acid sequence of the polypeptides as specified above. Suitable PCR conditions are well known in the art. As a template, DNA or cDNA from a mammal, in particular a human, may be used. Further, variants include polynucleotides comprising nucleic acid sequences which are at least 70%, at least 75%, at least 80%, at least 85%, at least 90%, at least 95%, at least 98% or at least 99% identical to the nucleic acid sequences detailed above. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences referred to above. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit (Needleman and Wunsch (J. Mol. Biol. 48; 443-453 (1970)) and Smith and Waterman (Adv. Appl. Math. 2; 482-489 (1981)), which are part of the GCG software packet (Genetics Computer Group, 575 Science Drive, Madison, Wisconsin, USA 53711 (1991)), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. A polynucleotide comprising a fragment of any of the aforementioned nucleic acid sequences is also encompassed as a polynucleotide, preferably as specified herein above. A fragment as meant herein, also preferably, may comprise at least 50, at least 100, at least 250 or at least 500 consecutive nucleotides of any one of the aforementioned nucleic acid sequences. The polynucleotides either essentially consist of the aforementioned nucleic acid sequences or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. The polynucleotide shall be provided, preferably, either as an isolated polynucleotide (i.e. isolated from its natural context) or in genetically modified form. The polynucleotide, preferably, is DNA, including cDNA, or RNA, preferably is mRNA. The term encompasses single—as well as double-stranded polynucleotides, preferably relates to single-stranded polynucleotides. Moreover, preferably comprised are also chemically modified polynucleotides including naturally occurring modified polynucleotides such as glycosylated or methylated polynucleotides or artificially modified ones such as biotinylated polynucleotides. Preferably, the polynucleotide has a length of from 10 bp to 5 Mb, more preferably of from 10 bp to 100 kb, still more preferably of from 50 bp to 20 kb, most preferably of from 100 bp to 10 kb.

The term “oligonucleotide”, as used herein, is understood by the skilled person. The term preferably relates to a nucleic acid molecule comprising of from 10 to 100, preferably of from 12 to 50, more preferably of from 14 to 25, most preferably about 18, nucleobases. The oligonucleotide referred to herein has the activity of binding to a polynucleotide comprising the nucleic acid sequence of SEQ ID NO:1, binding preferably referring to specific binding, in particular specific binding under standard conditions, more preferably under stringent conditions as specified elsewhere herein; also preferably, binding relates to in vivo binding, i.e. binding under conditions in a living cell. Preferably, the oligonucleotide comprises, more preferably consists of, a stretch of at least 10, preferably at least 12, more preferably at least 14 continuous bases essentially complementary to SEQ ID NO: 1, preferably to nucleotides 1 to 1000 of SEQ ID NO:1, more preferably to nucleotides 50 to 600 of SEQ ID NO:1, most preferably to nucleotides 100 to 300 of SEQ ID NO: 1. More preferably, the oligonucleotide comprises a stretch of from 10 to 100, preferably of from 12 to 50, more preferably of from 14 to 25, most preferably about 18, continuous bases essentially complementary to SEQ ID NO: 1, preferably to nucleotides 1 to 1000 of SEQ ID NO:1, more preferably to nucleotides 50 to 600 of SEQ ID NO:1, most preferably to nucleotides 100 to 300 of SEQ ID NO:1. In a preferred embodiment, the oligonucleotide comprises, more preferably consists of, a stretch of at least 10, preferably at least 12, more preferably at least 14 continuous bases essentially complementary to nucleotides 140 to 200 of SEQ ID NO: 1. Still more preferably, the oligonucleotide consists of a stretch of from 10 to 100, preferably of from 12 to 50, more preferably of from 14 to 25, most preferably about 18, continuous bases essentially complementary to SEQ ID NO:1, preferably to nucleotides 1 to 1000 of SEQ ID NO:1, more preferably to nucleotides 50 to 600 of SEQ ID NO: 1, most preferably to nucleotides 100 to 300 of SEQ ID NO:1. Most preferably, the oligonucleotide comprises, more preferably consists of, a stretch of from 10 to 100, preferably of from 12 to 50, more preferably of from 14 to 25, most preferably about 18, continuous bases essentially identical to SEQ ID NO:2, more preferably of SEQ ID NO:3. Moreover, the term oligonucleotide further encompasses variants of the indicated specific oligonucleotides. The oligonucleotide variants, preferably, comprise a nucleic acid sequence characterized in that the sequence can be derived from the aforementioned specific nucleic acid sequences by at least one nucleotide substitution, addition and/or deletion, wherein the variant oligonucleotides shall still have the binding property as indicated. Moreover, also encompassed are oligonucleotides which comprise nucleic acid sequences which are at least 70%, at least 75%, at least 80%, at least 85%, at least 90%, at least 95%, at least 98% or at least 99% identical to the specific nucleic acid sequences referred to above. The percent identity values are, preferably, calculated over the entire nucleic acid sequence region, preferably as specified herein above for the polynucleotides specified herein. As the skilled person understands, the parameters of the determination algorithms may be adjusted specifically for short sequence comparisons. The oligonucleotides either essentially consist of the aforementioned nucleic acid sequences or comprise the aforementioned nucleic acid sequences; thus, the oligonucleotides may contain further nucleic acid sequences as well. More preferably, however, the oligonucleotides consist of the aforementioned nucleic acid sequences. The oligonucleotide shall be provided, preferably, either as an isolated oligonucleotide (i.e. isolated from its natural context) or in genetically modified form. The oligonucleotide, preferably, is DNA, including cDNA, or RNA, more preferably is DNA. The term encompasses singleas well as double-stranded oligonucleotides, preferably relates to single-stranded molecules. Moreover, preferably comprised are also chemically modified oligonucleotides including naturally occurring modified oligonucleotides such as glycosylated or methylated oligonucleotides or artificially modified ones such as biotinylated oligonucleotides. Preferably, the oligonucleotide is modified to decrease its degradation rate in a cell; appropriate modifications are known in the art and include, in particular, replacing of one or more, preferably at least two, more preferably at least three nucleotides by phosphorothioate-nucleotides. Preferably, the oligonucleotide comprises, preferably consists of, the nucleic acid sequence of any one of SEQ ID NOs:4 to 17, still more preferably comprises, preferably consists of, the nucleic acid sequence of any one of SEQ ID NOs: 12 to 17, more preferably 14 to 17, most preferably 15 or 17. Also preferably, the oligonucleotide is comprised in a composition, preferably a pharmaceutical composition. In a preferred embodiment, the oligonucleotide comprises, preferably consists of, the nucleic acid sequence of any one of SEQ ID NOs:4 to 13 or 15 to 17, or consists of the nucleic acid sequence of SEQ ID NO:14. In a preferred embodiment, the oligonucleotide comprises, preferably consists of, the nucleic acid sequence of any one of SEQ ID NOs: 12 to 17 and 65, 70, 71, 75, 81, 83, 87-89, more preferably 14 to 17 and 65, 70, 71, 75, 81, 83, 87-89, most preferably 15, 17, 65, 70, 71, 75, 81, 83, 87, 88, or 89.

The term “composition”, as used herein, relates to any composition of matter comprising the indicated constituent(s). Preferably, the composition is a pharmaceutical composition.

The term “pharmaceutical composition”, as used herein, relates to a composition comprising the compound of the present invention and optionally one or more pharmaceutically acceptable carrier, wherein the constituents of the pharmaceutical composition are provided in a pharmaceutically acceptable form, i.e. in a form being not deleterious to a recipient thereof. In particular, the compounds of the present invention can be formulated as pharmaceutically acceptable salts or solutions. Preferred acceptable salts are acetate, methylester, HCl, sulfate, chloride, and the like. The pharmaceutical compositions are, preferably, administered topically or systemically, more preferably systemically. Suitable routes of administration conventionally used for drug administration are oral, intravenous, subcutaneous, or parenteral administration as well as inhalation. Preferably, administration is intracerebral, epidural, and/or intracerebroventricular. However, depending on the nature and mode of action of a compound, the pharmaceutical compositions may be administered by other routes as well. Moreover, the compounds can be administered in combination with other drugs either in a common pharmaceutical composition or as separated pharmaceutical compositions as specified elsewhere herein, wherein said separated pharmaceutical compositions may be provided in form of a kit of parts. Preferably, the combined preparation is an extended release preparation with regard to one or more of the compounds.

The compounds are, preferably, administered in conventional dosage forms prepared by combining the drugs with standard pharmaceutical carriers according to conventional procedures. These procedures may involve mixing, granulating and compressing or dissolving the ingredients as appropriate for the desired preparation. It will be appreciated that the form and character of the pharmaceutically acceptable carrier or diluent is dictated by the amount of active ingredient with which it is to be combined, the route of administration and other well-known variables. The carrier(s) must be acceptable in the sense of being compatible with the other ingredients of the formulation and being not deleterious to the recipient thereof. The pharmaceutical carrier employed may be, for example, a solid, a gel or a liquid. Exemplary of solid carriers are lactose, terra alba, sucrose, talc, gelatin, agar, pectin, acacia, magnesium stearate, stearic acid, and the like. Exemplary liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania. The diluent(s) is/are selected so as not to affect the biological activity of the compound or compounds. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers, reactive oxygen scavengers, and the like.

A therapeutically effective dose refers to an amount of the compounds to be used in a pharmaceutical composition of the present invention which prevents, ameliorates or treats the symptoms accompanying a disease or condition referred to in this specification. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. A typical dose can be, for example, in the range of from 1 μg to 1500 mg; however, doses below or above this exemplary range are envisioned, especially considering the aforementioned factors. Generally, the regimen as a regular administration of the pharmaceutical composition should be in the range of 100 ng to 100 mg units per day. If the regimen is a continuous infusion, it should also be in the range of 100 ng to 100 mg units per kilogram of body weight per minute, respectively. Preferably, extended release preparations of each drug are injected from once per 1 week to once per 2 months or even at longer intervals. Progress can be monitored by periodic assessment. Preferred doses and concentrations of the compounds of the present invention are specified elsewhere herein.

The pharmaceutical compositions and formulations referred to herein are, preferably, administered at least once, e.g. in case of extended release formulations, in order to treat or ameliorate or prevent a disease or condition recited in this specification. However, the said pharmaceutical compositions may be administered more than one time, for example from one to four times daily up to a non-limited number of days. Also some compounds with a short clearance time may be applied as infusion in blood stream to provide effective dose in whole body during long treatment time.

Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adopted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient.

Advantageously, it was found in the work underlying the present invention that the oligonucleotides of the present invention can increase the amount of Shank3 protein present in cells compared to controls. Without wishing to be bound by theory, it is assumed that the oligonucleotides prevent Shank3 mRNA from being degraded, thus increasing the amount of protein produced from each mRNA molecule.

The definitions made above apply mutatis mutandis to the following. Additional definitions and explanations made further below also apply for all embodiments described in this specification mutatis mutandis.

The present invention also relates to the oligonucleotide of the invention for use in medicine; and to the oligonucleotide of the invention for use in treatment and/or prevention of Shank3 deficiency, preferably for use in treatment and/or prevention of Phelan-McDermid syndrome. The present invention also relates to a pharmaceutical composition comprising the oligonucleotide of the present invention, preferably for use in medicine, preferably for use in treatment and/or prevention of Shank3 deficiency, preferably for use in treatment and/or prevention of Phelan-McDermid syndrome. The present invention also relates to a use of an oligonucleotide of the present invention for the manufacture of a pharmaceutical composition, in particular a pharmaceutical composition for treatment and/or prevention of Shank3 deficiency, preferably for use in treatment and/or prevention of Phelan-McDermid syndrome

The term “treatment” refers to an amelioration of a disease or disorder referred to herein or the symptoms accompanied therewith to a significant extent. Said treating as used herein also includes an entire restoration of the health with respect to the diseases or disorders referred to herein. It is to be understood that treating as used in accordance with the present invention may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 20%, more preferably at least 50%, even more preferably at least 60%, still more preferably at least 70%, still more preferably at least 80%, most preferably at least 90% of the subjects of a given cohort or population. As will be understood by the skilled person, effectiveness of treatment is dependent on a variety of factors including, e.g. disease stage and severity. As will be also understood, the term treatment includes measures preventing and/or delaying progression of a disease or disorder referred to herein.

The term “preventing” refers to retaining health with respect to the diseases or disorders referred to herein for a certain period of time in a subject. It will be understood that said period of time is dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification. As used herein, the term prevention, preferably, includes measures of gene therapy, as described elsewhere herein. Thus, the subject receiving preventive measures may be free of any symptoms of disease, but may be identified e.g. by gene analysis to have an increased risk of developing a disease or disorder referred to herein.

The term “Shank3 deficiency” is known to the skilled person to relate to any disease or disorder caused or aggravated at least in part by decreased expression of Shank3 in cells of a subject, causing morphological and neurological manifestations. Thus, preferably, the Shank3 deficiency is a synaptopaty, in particular a synaptopathy including loss of excitatory synapses; thus, preferably, the Shank3 deficiency is Alzheimer disease, amyotrophic lateral sclerosis, and/or apoplexy, in particular stroke. Preferably, the Shank3 deficiency is Phelan-McDermid syndrome, also known as 22q13 deletion syndrome. Symptoms of Phelan-McDermid syndrome are known in the art, the most frequent being global developmental delay, intellectual disability, speech, motor, and gait abnormalities. Diagnosis of Phelan-McDermid syndrome is usually established by combined genetic, cognitive and behavioral assessment. Shank3 deficiency can also be determined by determining Shank3 expression in cells of a subject, e.g. by a method as specified herein in the examples.

As used herein, the term “subject” relates to a vertebrate. Preferably, the subject is a mammal, more preferably, a mouse, rat, cat, dog, hamster, guinea pig, sheep, goat, pig, cattle, or horse. Still more preferably, the subject is a primate. Most preferably, the subject is a human. Preferably, the subject is known or suspected to be afflicted with or having an increased risk of becoming afflicted with Shank3 deficiency, in particular with Phelan-McDermid syndrome.

Preferably, treating and/or preventing Shank3 deficiency comprises contacting a subject with an oligonucleotide as specified herein above; more preferably, treating and/or preventing Shank3 deficiency comprises contacting neurons of said subject with said oligonucleotide; also preferably, treating and/or preventing comprises contacting said subject, preferably said neurons, with a plurality of non-identical oligonucleotides s specified herein above and/or with one or more constructs causing expression of oligonucleotides as specified herein below.

The present invention also relates to an expression construct, preferably comprised in an expression vector, encoding the oligonucleotide according to the present invention.

The term “expression construct”, as used herein, relates to a nucleic acid molecule encoding an oligonucleotide as specified herein and comprising at least one nucleic acid sequence causing the oligonucleotide to be expressed in a host cell. Preferably, in case the expression construct is a DNA, the nucleic acid sequence causing the oligonucleotide to be expressed preferably is a promoter, more preferably an RNA-polymerase promoter. The skilled person is able to select an appropriate promoter according to purpose; preferably, the promoter is an inducible promoter, more preferably, the promoter is a constitutive promoter. Also preferably, the promoter is a cell-type specific promoter, in particular a neuron specific promoter. More preferably, in case the expression construct is an RNA, the expression construct comprises at least one reverse transcriptase initiation site, i.e. preferably, from the RNA expression construct a DNA oligonucleotide as specified above is expressed in a host cell comprising a reverse transcriptase. Thus, preferably, the expression construct, preferably, further comprises an expressible reverse transcriptase gene.

The present invention further relates to a host cell, preferably a neuronal host cell, comprising the oligonucleotide as specified herein and/or an expression construct as specified herein. The term “host cell”, as used herein, relates to a eukaryotic cell, preferably a vertebrate cell, more preferably a mammalian cell, most preferably, a human cell. Preferably, the host cell is a neuronal host cell. Preferably, the host cell comprises at least one polynucleotide polymerase, preferably a reverse transcriptase and/or an RNA polymerase. More preferably, said polynucleotide polymerase initiates polymerization at a nucleic acid sequence comprised in the expression construct, preferably at the reverse transcriptase initiation site and/or the promoter.

The present invention also relates to a kit comprising an oligonucleotide as specified herein and a means of administration.

The term “kit”, as used herein, refers to a collection of the aforementioned compounds, means or reagents which may or may not be packaged together. The components of the kit may be comprised by separate vials (i.e. as a kit of separate parts) or provided in a single vial. Moreover, it is to be understood that the kit of the present invention, preferably, is to be used for practicing the methods referred to herein elsewhere. It is, preferably, envisaged that all components are provided in a ready-to-use manner for practicing the methods referred to above. Further, the kit, preferably, contains instructions for carrying out said methods. The instructions can be provided by a user's manual in paper or electronic form. In addition, the manual may comprise instructions for administration and/or dosage instructions for carrying out the aforementioned methods using the kit of the present invention. As will be understood from the above, the description of the kit comprising oligonucleotides, preferably, relates to a kit comprising corresponding expression constructs mutatis mutandis.

Preferably, the kit comprises the oligonucleotide as specified herein and a means of administration. Means of administration are all means suitable for administering the oligonucleotide and/or the expression construct to a subject. Thus, preferably, a means of administration is any arbitrary means configured and/or suitable for bringing the oligonucleotide and/or the expression construct into the body of a subject and/or into a host cell of said subject. The means of administration may include a delivery unit for the administration of the compound or composition and a storage unit for storing said compound or composition until administration. However, it is also contemplated that the means of the current invention may appear as separate devices in such an embodiment and are, preferably, packaged together in said kit. Preferred means for administration are those which can be applied without the particular knowledge of a specialized technician. Preferably, the means for administration is a syringe, more preferably with a needle, comprising the compound or composition of the invention. In another preferred embodiment, the means for administration is an intravenous infusion (IV) equipment comprising the compound or composition. In still another preferred embodiment the means for administration is an inhaler comprising the compound of the present invention, wherein, more preferably, said compound is formulated for administration as an aerosol. Also preferably, the means for administration is a chemical reagent causing or improving uptake of the oligonucleotide and/or the expression construct into a host cell. Thus the means for administration may be a transfection reagent. Appropriate reagents are known in the art.

The present invention also relates to a device comprising an oligonucleotide as specified herein and/or an expression construct as specified herein.

The device may be a means of administration as specified herein above; the device may, however, also be a device configured for performing one of the in vitro methods of the present invention, in particular a pipetting and/or incubation device configured for performing the method for determining whether a subject suffering from Shank3 deficiency is amenable to a therapy with an oligonucleotide as specified herein below.

The present invention also relates to a method for increasing Shank3 expression in a host cell, comprising contacting said host cell with an oligonucleotide as specified herein, and, thereby, increasing Shank 3 expression.

The method for increasing Shank3 expression of the present invention, preferably, is an in vitro method. Moreover, it may comprise steps in addition to those explicitly mentioned above. For example, further steps may relate, e.g., to providing an oligonucleotide, or incubating said host cells after contacting. Moreover, one or more of said steps may be performed by automated equipment.

The present invention also relates to a method of treating Shank3 deficiency in a subject, comprising contacting said subject with an oligonucleotide as specified herein, thereby treating Shank3 deficiency.

The present invention further relates to a method for determining whether a subject suffering from Shank3 deficiency is amenable to a therapy with an oligonucleotide as specified herein, comprising
a) contacting a cell sample of said subject with said oligonucleotide;
b) determining the amount of Shank3 polypeptide in said cell sample;
c) comparing the amount of Shank3 polypeptide determined in step b) to a reference; and
d) determining whether a subject suffering from Shank deficiency is amenable to a therapy based on the result of comparison step c).

The method for determining whether a subject suffering from Shank3 deficiency is amenable to a therapy of the present invention, preferably, is an in vitro method. Moreover, it may comprise steps in addition to those explicitly mentioned above. For example, further steps may relate, e.g., to providing a cell sample for step a)), and/or incubation of the cells of step a) and before step b), e.g. to allow Shank3 expression to increase. Preferably, in the method comprises further steps al) providing a control cell sample not contacted with said oligonucleotide and b1) determining the amount of Shank3 polypeptide in said control cell sample, and step c) is comparing the amount of Shank3 polypeptide determined in step b) to the amount of Shank3 polypeptide determined in step b1); preferably, in such case, a subject suffering from Shank deficiency is amenable to a therapy is identified in case the amount of Shank3 determined in step b) is higher, preferably significantly higher, than the amount of Shank3 determined in step b1). Moreover, one or more of said steps may be performed by automated equipment.

The term “cell sample”, as used herein, refers to a sample comprising intact cells, preferably of separated cells, of a tissue or of an organ of a subject. Also included as a sample is a sample of cultured cells. Methods for obtaining cell samples are known in the art and include in particular biopsies, excisions, and blood taking. Preferably, the cell sample is a sample comprising blood cells, also preferably a sample comprising neuronal cells.

The term “reference” is known to the skilled person to refer to a discriminator which allows identification of a subject suffering from Shank3 deficiency being amenable to a therapy with an oligonucleotide as specified herein. Such a discriminator may be a target value of a Shank3 concentration or of a relative amount in a host cell. Preferably, the reference is a quantitative indicator, more preferably is a value, e.g. a threshold value, is a range, e.g. a range of values, is a score, or is any other value or range deemed appropriate by the skilled person. Preferably, the reference is determined based on (i) a control cell sample of said subject not contacted to said oligonucleotide; (ii) a population of apparently healthy subjects; (iii) a population of subjects known not to suffer from Shank3 deficiency; (iv) a population of subjects known to suffer from Shank3 deficiency. Preferably, the reference is obtained from a subject or a group of subjects suffering from Shank3 deficiency; in such case, in case the reference is e.g. a threshold value, a subject suffering from Shank deficiency amenable to a therapy as specified is identified in case the amount of Shank3 determined in step b) is higher than the reference. Also preferably, the reference is obtained from a subject or a group of subjects known not to suffer from Shank3 deficiency; in such case, in case the reference is e.g. a threshold value, a subject suffering from Shank deficiency amenable to a therapy as specified is identified in case the amount of Shank3 determined in step b) is equal to or higher than the reference; in this latter case, the reference may also be based on a population of apparently healthy subjects. Thus, preferably, the reference is (I) an amount of Shank3 based on any one of (i) to (iii) as specified above and an amount essentially increased or higher than said reference is indicative of a subject amenable to said therapy; (II) an amount of Shank3 based on (iv) as specified above and an amount essentially identical or lower than said reference is indicative of a subject not amenable to said therapy; (III) a reference range determined based on any one of (i) to (iv) as specified above or a combination of any of (i) to (iv) as specified above, preferably based on any one of (i) to (iii) as specified above and (iv) as specified above, and an amount within said reference range is indicative of a subject amenable to said therapy; (IV) a score calculated comprising any one of (i) to (iv) as specified above.

In view of the above, the following embodiments are particularly envisaged:

Embodiment 1: An oligonucleotide specifically binding to a polynucleotide comprising the nucleic acid sequence of SEQ ID NO:1, preferably comprising nucleotides 1 to 1000 of SEQ ID NO:1, more preferably nucleotides 50 to 600 of SEQ ID NO:1, most preferably nucleotides 100 to 300 of SEQ ID NO:1.

Embodiment 2: The oligonucleotide of embodiment 1, wherein said oligonucleotide comprises a stretch of at least 10, preferably at least 12, more preferably at least 14 continuous bases essentially complementary to SEQ ID NO:1, preferably to nucleotides 1 to 1000 of SEQ ID NO:1, more preferably to nucleotides 50 to 600 of SEQ ID NO:1, most preferably to nucleotides 100 to 300 of SEQ ID NO:1.

Embodiment 3: The oligonucleotide of embodiment 1 or 2, wherein said oligonucleotide comprises a stretch of from 10 to 100, preferably of from 12 to 50, more preferably of from 14 to 25, most preferably about 18, continuous bases essentially complementary to SEQ ID NO: 1, preferably to nucleotides 1 to 1000 of SEQ ID NO: 1, more preferably to nucleotides 50 to 600 of SEQ ID NO:1, most preferably to nucleotides 100 to 300 of SEQ ID NO: 1.

Embodiment 4: The oligonucleotide of any one of embodiments 1 to 3, wherein said oligonucleotide consists of a stretch of from 10 to 100, preferably of from 12 to 50, more preferably of from 14 to 25, most preferably about 18, continuous bases essentially complementary to SEQ ID NO:1, preferably to nucleotides 1 to 1000 of SEQ ID NO:1, more preferably to nucleotides 50 to 600 of SEQ ID NO: 1, most preferably to nucleotides 100 to 300 of SEQ ID NO:1.

Embodiment 5: The oligonucleotide of any one of embodiments 1 to 4, wherein said oligonucleotide comprises a stretch of from 10 to 100, preferably of from 12 to 50, more preferably of from 14 to 25, most preferably about 18, continuous bases essentially identical to SEQ ID NO:2, more preferably SEQ ID NO:3.

Embodiment 6: The oligonucleotide of any one of embodiments 1 to 5, wherein said oligonucleotide consists of a stretch of from 10 to 100, preferably of from 12 to 50, more preferably of from 14 to 25, most preferably about 18, continuous bases essentially identical to SEQ ID NO:2.

Embodiment 7: The oligonucleotide of any one of embodiments 1 to 6, wherein said oligonucleotide consists of from 10 to 100, preferably of from 12 to 50, more preferably of from 14 to 25, most preferably about 18 nucleotides.

Embodiment 8: The oligonucleotide of any one of embodiments 1 to 7, wherein said oligonucleotide comprises, preferably consists of, the nucleic acid sequence of any one of SEQ ID NOs: 4 to 17, preferably 12 to 17.

Embodiment 9: The oligonucleotide of any one of embodiments 1 to 8, wherein said oligonucleotide comprises, preferably consists of, the nucleic acid sequence of any one of SEQ ID NOs: 14 to 17, preferably 15 or 17.

Embodiment 10: The oligonucleotide of any one of embodiments 1 to 9, wherein said oligonucleotide comprises deoxynucleotides, preferably is a DNA oligonucleotide and/or comprises phosphorotioate nucleotides.

Embodiment 11: The oligonucleotide of any one of embodiments 1 to 10, wherein said oligonucleotide is coupled, preferably covalently coupled, to at least one of a membrane transport signal and a nuclear localization signal.

Embodiment 12: The oligonucleotide of any one of embodiments 1 to 11, comprised in a composition, preferably a pharmaceutical composition.

Embodiment 13: An oligonucleotide of any one of embodiments 1 to 12 for use in medicine.

Embodiment 14: An oligonucleotide of any one of embodiments 1 to 12 for use in treatment and/or prevention of Shank3 deficiency, preferably for use in treatment and/or prevention of Phelan-McDermid syndrome.

Embodiment 15: The oligonucleotide for use of embodiment 14, wherein said use comprises contacting neurons of a subject with said oligonucleotide.

Embodiment 16: The oligonucleotide for use of embodiment 14 or 15, wherein said use comprises contacting neurons of a subject suffering from said Shank3 deficiency with a construct causing expression of said oligonucleotide in said neurons, preferably with the expression construct according to embodiment 19.

Embodiment 17: The oligonucleotide for use of embodiment 15 or 16, wherein said use comprises contacting said neurons with a plurality of non-identical oligonucleotides according to any one of embodiments 1 to 12.

Embodiment 18: A pharmaceutical composition comprising the oligonucleotide of any one of embodiments 1 to 12.

Embodiment 19: An expression construct, preferably comprised in an expression vector, encoding the oligonucleotide according to any one of embodiments 1 to 12.

Embodiment 20: A host cell, preferably a neuronal host cell, comprising the oligonucleotide according to any one of embodiments 1 to 12 and/or the expression construct according to embodiment 19.

Embodiment 21: A kit comprising an oligonucleotide according to any one of embodiments 1 to 12 and/or an expression construct according to embodiment 19; and a means of administration thereof.

Embodiment 22: A device comprising an oligonucleotide according to any one of embodiments 1 to 12 and/or an oligonucleotide according to embodiment 19 and/or an expression construct Embodiment 23: A method for increasing Shank3 expression in a host cell, comprising contacting said host cell with an oligonucleotide according to any one of embodiments 1 to 12, and, thereby, increasing Shank 3 expression.

Embodiment 24: The method of embodiment 23, wherein said method is an in vitro method.

Embodiment 25: A method of treating Shank3 deficiency in a subject, comprising contacting said subject with an oligonucleotide according to any one of embodiments 1 to 12, thereby treating Shank3 deficiency.

Embodiment 26. The method of embodiment 25, wherein neurons of said subject are contacted with said oligonucleotide.

Embodiment 27: A method for determining whether a subject suffering from Shank3 deficiency is amenable to a therapy with an oligonucleotide according to any one of embodiments 1 to 12, comprising
a) contacting a cell sample of said subject with said oligonucleotide;
b) determining the amount of Shank3 polypeptide in said cell sample;
c) comparing the amount of Shank3 polypeptide determined in step b) to a reference; and
d) determining whether a subject suffering from Shank deficiency is amenable to a therapy based on the result of comparison step c).

Embodiment 28: The method of embodiment 27, wherein said reference is determined based on
(i) a control cell sample of said subject not contacted to said oligonucleotide;
(ii) a population of apparently healthy subjects
(iii) a population of subjects known not to suffer from Shank3 deficiency;
(iv) a population of subjects known to suffer from Shank3 deficiency.

Embodiment 29: The method of embodiment 28, wherein said reference is
(I) an amount of Shank3 based on any one of (i) to (iii) of embodiment 28 and wherein an amount essentially increased or higher than said reference is indicative of a subject amenable to said therapy;
(II) an amount of Shank3 based on (iv) of embodiment 29 and wherein an amount essentially identical or lower than said reference is indicative of a subject not amenable to said therapy;
(III) a reference range determined based on any one of (i) to (iv) of embodiment 28 or a combination of any of (i) to (iv) of embodiment 28, preferably based on any one of (i) to (iii) of embodiment 28 and (iv) of embodiment 28, and wherein an amount within said reference range is indicative of a subject amenable to said therapy;
(IV) a score calculated comprising any one of (i) to (iv) of embodiment 28.

Embodiment 30: Use of an oligonucleotide according to any one of embodiments 1 to 12 for the manufacture of a medicament for the treatment and/or prevention of Shank3 deficiency.

All references cited in this specification are herewith incorporated by reference with respect to their entire disclosure content and the disclosure content specifically mentioned in this specification.

### FIGURE LEGENDS

FIG. 1: Transfection of human CTRL and PMDS iPSC with 50 bp long antisense oligonucleotides. SHANK3 protein levels of the 110 kDa isoform after transfection were detected via western blotting and normalized on the SHANK3 levels in scrambled transfected iPSC. (A/C) SHANK3 levels in transfected CTRL1/PMDS2 iPSC as detected in western blot (B/D). Some AONs increase the SHANK3 expression whereas others decrease it compared to scrambled transfected samples.

FIG. 2: Transfection of iPSC and motoneurons using the most effective AONs from the screening. SHANK3 protein levels after transfection were detected via western blotting and normalized on the SHANK3 levels in scrambled transfected iPSC. SHANK3 levels in transfected CTRL1/PMDS2 iPSC (A) as detected in western blot (B) and in transfected CTRL1/PMDS2 motoneurons (C) again as detected in western blot (D). Bars represent overall SHANK3 expression as mean of triplicates±standard error of the mean (SEM) (one-way ANOVA with p<0.05≙*, p=0.01≙**, p=0.001≙***).

FIG. 3: SHANK3 expression in transfected iPSC using 18 bp AONs generated from the most effective 50 bp AONs. SHANK3 protein levels after transfection were detected via western blotting and normalized on the scrambled transfected iPSC. SHANK3 levels in transfected CTRL1/PMDS2 iPSC as detected in western blot. Bars represent overall SHANK3 expression as mean of triplicates+standard error of the mean (SEM) (one-way ANOVA with p<0.05≙*, p=0.01≙**, p=0.001≙***).

FIG. 4: SHANK3 levels in motoneurons after transfection with promising 18 bp AONs. d42 motoneurons from CTRL1 and PMDS2 were transfected and SHANK3 levels were determined via western blotting. Bars represent overall SHANK3 expression as mean of triplicates+standard error of the mean (SEM) (one-way ANOVA with p<0.05≙*, p=0.01≙**, p=0.001≙***).

FIG. 5: SHANK3 expression in transfected NPC-motoneurons using the most effective 18 bp AONs. Bars represent overall SHANK3 expression as mean of triplicates+standard error of the mean (SEM) (one-way ANOVA with p<0.05≙*, p=0.01≙**, p=0.001≙***).

FIG. 6: SHANK3 expression as detected in ICC in transfected motoneurons. NPC-motoneurons stained with DAPI (nuclei), SHANK3, Biotin, and NEFH (neurofilaments). Merge of all channels and SHANK3 are shown. Bars represent mean+SEM (one-way ANOVA with p<0.05≙*, p=0.01≙**, p=0.001≙***). Scale bars 20 μm.

FIG. 7: SHANK3 protein levels in transfected NPC motoneurons. Protein levels detected via Western Blotting. Expression has been normalized on scrambled and all isoforms have been pooled. Bars represent mean+SEM (n=3, one-way ANOVA with p<0.05≙*, p=0.01≙**, p=0.001≙***, p=0.0001≙****).

FIG. 8: SHANK3 expression as detected in ICC in transfected NPC-motoneurons. NPC-motoneurons stained with DAPI (nuclei), SHANK3, Biotin, and MAP2 (neurofilaments). Quantification of nuclear SHANK3 levels. Bars represent mean. Scale bar 20 μm.

FIG. 9: SHANK3 expression as detected in ICC in transfected motoneurons. (A) Motoneurons stained with DAPI, SHANK3, Biotin and MAP2. (B), (C) Quantification of nuclear SHANK3 levels. (B) shows normalized SHANK3 levels detected in each cell line, (C) shows pooled data for controls and PMDS-patients. Bars represent mean+SEM (n=3, one-way ANOVA with p<0.05 [symbol] *, p=0.01 [symbol] **, p=0.001 [symbol] ***, p=0.0001 [symbol] ****). Scale bar 10 μm.

FIG. 10: Transfection of human CTRL and PMDS iPSC with shifted 18 bp long antisense oligonucleotides. SHANK3 protein levels of the 300 kDa isoform after transfection were detected via western blotting and normalized on the SHANK3 levels in untreated iPSCs. (A), (B) SHANK3 levels in transfected CTRL1 (A) or PMDS2 (B) iPSC as detected in western blot.

The following Examples shall merely illustrate the invention. They shall not be construed, whatsoever, to limit the scope of the invention.

### Example 1: Methods

All reagents used, their manufacturers and catalogue numbers can be found in Table 1.

TABLE 1
Reagents

| Material | Manufacturer | Catalogue Number |
|---|---|---|
| Accutase | StemPro Sigma | A6964-500ML |
| Acrylamida | Serva | A3678-1006 |
| AmershamTM Hybond PVDF transfer | GE Healthcare | |
| membrane | | |
| Ammonium persulfate | Sigma-Aldrich | A3678-25G |
| Antibiotic-Antimycotic, 100X | Invitrogen | 15240062 |
| Apo-Transferrin, human recombinant | Sigma-Aldrich | T2036-1G |
| Ascorbic Acid | Sigma-Aldrich | A4403 |
| B27 without Vit A | Gibco | 12587 |
| β-Mercaptoethanol | Invitrogen | 21985023 |
| BDNF, human recombinant | Peprotech | 540-02 |
| Bromophenol blue | | |
| BSA—Bovine Serum Albumin | CarlRoth | 8076.3 |
| cAMP | Sigma-Aldrich | 16980-89-5 |
| Cellstar cell culture plate | Greiner Bio-One | |
| CHIR99021 | Stemcell | 72054 |
| | Technologies | |
| Dispase, 5 U/ml | Stemcell | 7913 |
| | Technologies | |
| DMEM/F12 + Glutamax | Gibco | 31331-028 |
| Dorsomorphin | Tocris | 3093 |
| Dithiothreitol (DTT) | | |
| Ethylenediaminetetraacetic acid (EDTA) | | |
| Ethanol absolute, more than 99.8% | Sigma-Aldrich | 32205-2.5L |
| FBS—Fetal Bovine Serum | Life Technologies | 10500-064 |
| Filtropur S sterile filters 0.2 μm | Sarstedt | |
| GDNF, human recombinant | Peprotech | 450-10 |
| Glycerol | | |
| Glycine | AppliChem | A1377, 1000 |
| Heparin Sodium Salt | Sigma-Aldrich | H3149 |
| Hormone Mix | | |
| IGF1, human recombinant | Peprotech | 100-11 |
| Insulin, human recombinant | SAFC | 91077C-1G |
| Isopropanol (70%) | VWR | 20.842.330 |
| Knockout DMEM with 4.5 g/L Glucose | Invitrogen | 10829018 |
| Knockout Serum Replacement | Invitrogen | 10828082 |
| Laminin, 2.0 mg/ml | Roche | 11243217001 |
| Lipofectamine ® 3000 (LF3000) | Thermo Fisher | L3000015 |
| | Scientific | |
| Matrigel ® Growth Factor Reduced | Life Sciences | 356231 |
| | Corning | |
| Matrigel, hESC qualified | Corning | 354277 |
| Methanol | Sigma-Aldrich | 32313-5L |
| μ-Dish 35 mm, high, ibiTreat, sterile | Ibidi | 81156 |
| mFreSR | Stemcell | 5855 |
| | Technologies | |
| mTeSR ®1 Kit | Stemcell | 5850 |
| | Technologies | |
| N2 supplement | Invitrogen | 17502048 |
| NaCl | Sigma-Aldrich | 31434-1KG-R |
| Na-Orthovanadate | | |
| Neurobasal | Invitrogen | 21103049 |
| NGS—Normal Goat Serum | Life Technologies | 16210072 |
| N,N,N′,N′-Tetramethylethylenediamin | | |
| (TEMED) | Sigma-Aldrich | T9281-25ML |
| Nonessential Amino Acids (NEEA) | Invitrogen | 10370021 |
| PBS −MgCl₂/−CaCl₂ | Life Technologies | 14190-094 |
| PBS +MgCl₂/+CaCl₂ | Life Technologies | 14040-091 |
| PFA | Carl Roth | 0335.3 |
| Phosphatase inhibitor | | |
| Pierce ® ECL Western Blotting Substrate | Thermo Scientific | 32106 |
| ProLong ® Gold Antifade Mountant with | Life Technologies | P36935 |
| DAPI | | |
| Protease inhibitor (complete) | Roche | 4693116001 |
| Pumorphamine (PU) | Calbiochem | 540220 |
| Poly-L-Ornithin | Sigma-Aldrich | P4957 |
| Progesterone | Sigma | P6149-1MG |
| Retinoic acid | Sigma Aldrich | R2625-100MG |
| Rock-Inhibitor | Selleck Chem | S1049 |
| SB-431542 | Stemcell | 72232 |
| | Technologies | |
| SDS ultrapure | Roth | 2326.2 |
| Spectra ™ Multicolor High Range Protein | Thermo Scientific | 26625 |
| Ladder | | |
| Sucrose | CarlRoth | 4621.1 |
| Tergitol NP-40 (NP40) | | |
| Tris molecular biology grade | AppliChem | A2264, 1000 |
| Triton X-100 | Carl Roth | 3051.3 |
| TrypLE Express | Life Technologies | 12604-021 |
| Tween-20 | Carl Roth | 9127.1 |
| Ultra-low attachment flask, 25 cm² | Corning Costar | |

### Example 2: iPSC and Differentiation

Human induced pluripotent stem cells (iPSC) were generated from hairs plucked with the root of PMDS patients and healthy controls using the protocol previously established by Linta et al. (2012), Stem Cells and Development, 21(6):965 to reprogram keratinocytes into iPSC. Two healthy control cell lines have been used as well as three cell lines generated from PMDS patients. Their deletion size, clinical phenotype and further information can be taken from Table 2.

TABLE 2
Patient characteristics

| iPSC | | | | |
|---|---|---|---|---|
| Line | Genotype | Gender | Diagnosis | Clinical phenotype |
| CTRL1 | Normal | ♀ | None | Healthy and unaffected |
| CTRL2 | Normal (all | ♀ | PMDS | Language delay and speech regression |
| | iPSC clones as | | (mosaic, | at the age of 6, no motor delay or |
| | well as the | | 55%) | relapse, auto aggressive behavior, |
| | keratinocytes | | | autism |
| | examined are | | | |
| | negative for the | | | |
| | 2.5-3 Mb | | | |
| | terminal 22q13 | | | |
| | deletion | | | |
| | detected in 55% | | | |
| | of the PMDS | | | |
| | mosaic patient) | | | |
| PMDS1 | 116 kb 22q13 | ♂ | PMDS | Neonatal hypotonia, language |
| | deletion | | | impairment, developmental delay |
| PMDS2 | 2574 kb 22q13 | ♀ | PMDS | Neonatal and persistent hypotonia, |
| | deletion | | | inclu-ding balance and coordination |
| | | | | problems and impairment of fine and |
| | | | | global motor skills, intellectual |
| | | | | disability, severe language delay |
| PMDS29 | 6 Mb deletion | ♂ | PMDS | Severe and persistent hypotonia (wheel- |
| | in chr. 22q13 | | | chair), medium to severe intellectual |
| | including | | | dis-ability, general developmental |
| | ARHGAP8, | | | delay, strabismus divergens, |
| | RABL2B and | | | macrocephaly, two-sided double |
| | SHANK3 | | | kidney, two-sided non descensus testes, |
| | | | | bicuspid aortic valve |

Human iPSC were cultured in an oxygen reduced atmosphere (5% O2) in an incubator at 37° C. with 5% CO2 in a feeder-free system on Matrigel-coated plates in mTeSR1 medium. All cell culture procedures were executed under sterile conditions. Every day, spontaneously differentiated cells were removed mechanically with a pipette tip followed by a washing step with DMEM-F12 to remove differentiated or dead cells. After washing, 1.5 ml of new mTeSR1 were added. When the cells reached a confluence of approximately 80% or large colony size, they were split in a ratio of 1:3 to 1:8 using Dispase and transferred to a new 6-well plate.

Motoneurons were generated using two different protocols. Motoneurons generated directly from iPSC following the protocol published by Hu & Zhang (2009), Nature Protocols 4(9): 1295, will be referred to as mn (Method 1), whereas motoneurons generated from neuronal progenitor cells (NPC) will be referred to as NPC-mn (Method 2). To differentiate human iPSC into mn for immunocytochemistry, we followed the protocol as published by Catanese and colleagues (2019), Autophagy 15(10): 1719.

To start a differentiation into mn for western blot analysis, at least six densely grown wells of iPSC were used. During the next 28 days, cells were treated as described in the following scheme previously published by Hu & Zhang (2009, loc. cit.).

### Method 1:

### Day 1: Generation of Embryoid Bodies

Human iPSCs were detached in a total of 10 ml of hESC medium (77% DMEM/F12, 20% KO-SR, 1% NEAA, 1% Antibiotic-Antimycotic, 1% B-mercaptoethanol) and transferred to a low attachment flask and 10 μM Rho-associated kinase (ROCK) inhibitor was added for the first 48 h. Embryoid bodies (EBs) formed after 1 day. Part of the medium was changed daily by letting the embryoid bodies sediment to the bottom of the flask and replacing 5-7 ml of the medium with new hESC medium.

### Day 4-8: Induction of Neuronal Differentiation

All further differentiation media are based on mn basal medium (96% DMEM/F12, 2% hormone mix, 1% NEAA, 1% Antibiotic-Antimycotic, 0.00004% Heparin (50 mg/ml)). Cells were left to sediment, then 7 ml of the old medium were removed and replaced by Diff. 1 medium (mn basal, 1:1000 GDNF, BDNF, IGF-1 and Vit. C, 1:10000 cAMP) including brain-derived neurotrophic factor (BDNF), glial-derived neurotrophic factor (GDNF) and insulin-like growth factor 1 (IGF1) along with Vitamin C (Vit. C) and cyclic adenosine monophosphate (cAMP). Part of the medium was replaced daily.

### Day 8: Plating of Cells and Formation of Neuronal Rosettes

One 12-well plate was coated with Laminin (1:50 in MN basal medium, 1 h, 37° C.). EBs were transferred into a falcon tube and centrifuged for 2 min at 100 g. The supernatant was discarded and the EBs carefully resuspended in 6 ml of Diff. 1 medium. 0.5 ml of the suspension containing the EBs were transferred to each well of the 12-well plate. After 24 h, 0.5 ml of Diff. 1 medium were added to each well.

### Day 10-14: Induction of Caudalization

Medium was changed to Diff. 2 medium containing retinoic acid (RA) (mn basal, 1:1000 GDNF, BDNF, IGF-1 and Vit. C, 1:10000 cAMP and RA) to induce caudalization. Medium was changed three times a week.

### Day 15: Suspension and Induction of Ventralization

The medium was removed and neuronal rosettes were rinsed off the plate with Diff. 3 medium containing pumorphamine (PU) (mn basal, 1:1000 GDNF, BDNF, IGF-1 and Vit. C, 1:10000 CAMP, RA and PU, 1:50 B27) to induce ventralization. Subsequently, they were transferred into a low attachment flask in suspension culture for the formation of neuronal spheres. The medium was changed three times a week.

### Day 28: Seeding of Neuronal Spheres and Maturation of Motoneurons

Firstly, ibidi μ-dishes were coated with Poly-L-Ornithine (PLO) for two h at 37° C. to increase surface hydrophobicity. Residual traces of toxic PLO were removed by washing three times with PBS+MgCl2/+CaCl2 before coating with Laminin as previously described. Afterwards, the Laminin was removed and 1.5 ml of Diff. 4 medium (mn basal, 1:1000 GDNF, BDNF, IGF-1 and Vit. C, 1:10000 cAMP and PU, 1:20000 RA, 1:50 B27) was added to each u-dish. The neuronal sphere suspension was transferred from the low attachment flask into a 15 ml tube and centrifuged at 100 g for two min. The supernatant was discarded and the neuronal spheres incubated with 2 ml of TrypLE for 2 min in a water bath with 37° C. to reduce neuronal sphere size. The reaction was stopped by the addition of DMEM-F12 and again, the cells were centrifuged and the supernatant discarded to remove residual enzyme. Neuronal spheres were resuspended in 500 μl Diff. 4 medium per dish and distributed evenly between the coated dishes. 1 ml of the medium was carefully exchanged once a week. Forty-two days after the final plating, mature motoneurons were obtained.

### Method 2:

To differentiate human iPSC into NPCs and then NPC-mn, the following protocol was used as previously published by Reinhardt et al. (2013), PLOS One 8(11):10.1371/annotation/6a917a2e-df4a-4ad9-99bb-6aa7218b833e. NPC basal medium consisted of 1:1 DMEM-F12 and Neurobasal with 1% Anti-Anti and all used media are based on the basal medium.

Day 0-1, hESC Medium

Human iPSC were detached as described previously, resuspended in hESC medium (10 μM SB-431542, 1 μM dorsomorphin, 3 μM CHIR99021, 0.5 μM PU) and further cultivation occurred in low-attachment flasks. Medium was changed daily.

Day 2-3, NPC d2-3 Medium

Old medium was removed completely and replaced by NPC d2-3 medium (1:200 N2, 1:100 B27, 10 μM SB-431542, 1 μM dorsomorphin, 3 μM CHIR99021, 0.5 μM PU), cells were cultivated further in low-attachment flasks.

Day 4-5, NPC d4-X+3 Medium

EBs were further cultivated in low-attachment flasks and medium was changed to NPC d4-X+3 medium (1:200 N2, 1:100 B27, 3 μM CHIR99021, 0.5 μM PU, 150 μM ascorbic acid).

Day 6, NPC d4-X+3 Medium

EBs were seeded completely to a Matrigel-coated 12-well plate (growth-factor reduced). Depending on the size of the EBs, 6-15 EBs were seeded in each well. On day 6, 0.5 ml medium should be used for each well for seeding. Therefore, plates were coated with 500 μl growth factor reduced Matrigel (1:100 in Knockout-DMEM). On day 7, 0.25 ml medium were added to each well. Since the EBs were not attached strongly to the plate after one day, the medium was not removed completely. After five splits the cultures were free of non-NPC.

Day 8-10 and Up to Day X (X Last Split Before the Start of Diff.), NPC d4-X+3 Medium

For the first split, EBs showing a neuro-epithelial outgrowth were chosen and two suitable wells were split 1:6 on a coated 12-well plate. EBs were digested with pre-warmed Accutase (15 min, 37° C.), the enzyme reaction was stopped by addition of 2 ml DMEM-F12 and the EBs were resuspended carefully with a pipette until no EBs were visible anymore. The turbid single-cell solution was transferred to a reaction tube and centrifuged for 2 min at 100 g. The supernatant was discarded and the pellet resuspended in NPC medium with supplements. A volume of 0.75 ml of the cell suspension was added to each well. At this step, NPCs can be frozen and to start a differentiation, NPCs should be unfrozen, passaged three times and then can be split as described below at “last split before differentiation”.

All other splits were carried out at a ratio of 1:6 once the cells reached a confluence of at least 90%. Cells were treated with pre-warmed Accutase as described above and seeded onto new 12-well plates. At the last split before differentiation, cells were again treated with Accutase and after centrifugation resuspended in 1 ml NPC medium in reaction tubes. A cell number of ˜50000 cells per well was used which equals ˜20000 cells/cm². For the counting of the cells the suspension was diluted 1:100 in DMEM-F12, counting was performed in a Neubauer Counting Chamber. To calculate the number of cells, the following formula was used:

cells/ml=cell number×dilution factor×10⁴

Day X-X+3, NPC d4-X+3 Medium

Cells were further cultivated on 12-well plates and medium was changed completely every two days. If a lot of dead cells were seen, a washing step was applied before adding the new medium.

Day X+4-X+5, NPC dX+4 5 Medium

The old medium was aspirated and replaced by NPC dX+4/5 medium (1:200 N2, 1:100 B27, 1 μM PU).

Day X+6-X+13, NPC dX+6 Medium

Medium was changed completely to NPC dX+6 medium (1:200 N2, 1:100 B27, 1 μM PU, 1 μM

RA) and a complete medium change was performed every two days (Monday, Wednesday, Fri-day). If many dead cells were visible, the cells were washed with DMEM-F12.

### Day X+14-End, NPC Maturation Medium

Medium was changed to NPC maturation medium (1:200 N2, 1:100 B27, 10 ng/μl BDNF and GDNF, 1 μM cAMP) for one day, then at day 15, cells were split, counted and seeded to a density of 50000 cells per well. The split was carried out onto 12-well plates. Twice a week half of the medium was removed and exchanged with fresh medium. After one month, mature NPC-mn were obtained.

Method for Differentiation into Mn for ICC:

To differentiate human iPSC into mn for ICC, the following protocol was used as previously published by Catanese and colleagues (2019). EBs were generated in supplemented hESC Medium (7.7 ml DMEM/F12+GlutaMAX, 2 ml KO-SR, 100 μl Anti-Anti, 100 μl NEAA, 100 μl Mercaptoethanol) and after plating, cells were kept in supplemented Neurobasal medium (480 ml DMEM/F12+GlutaMAX, 10 ml Hormone Mix, 5 ml Anti-Anti, 5 ml NEAA, 20 μl Heparin).

### Day 0

Cells were detached by Dispase (3 min) and cultivated in suspension in ultra-low attachment flasks T75 to form EBs in New hESC medium (10 ml hESC medium, 100 μl B27 without Vit. A, 50 μl N2, 10 μl of each 3 μM CHIR99021, 10 μM SB-431542 and 200 μM Vit. C, 5 μl 1 μM dorsomorphin, 1 μl 1 μM pumorphamine).

### Day 1

Complete Removal of the Old hESC Medium and Add New hESC Medium

### Day 3

Medium was switched to New Diff Medium (10 ml Neurobasal, 100 μl B27 without Vit. A, 50 μl N2, 10 μl of each 3 μM CHIR99021, 10 μM SB-431542, 10 μM BDNF, 10 μM GDNF, 10 UM IGF1 and 200 μM Vit. C, 5 μl 1 μM dorsomorphin, 1 μl 1 μM pumorphamine, 1 μl 500 μM cAMP, 1 μl 1 μM retinoic acid).

### Day 5+7

### Remove 5 ml of the Old Medium and Add 5 ml Fresh New Diff Medium

### Day 8:

Cells were counted (40.000 per ibidi) and plated (coating with Growth Factor Reduced Matrigel for 2 h at 37). Mn were then cultivated for 28 days, thereafter they were transfected, fixated and stained.

### Example 3: General Methods

Transfection with Lipofectamine®

For each approach, two reaction tubes were prepared. 50 μl DMEM-F12 was pipetted into each tube. To one 0.71% LF3000 was added, to the other tube 7.2% P3000 reagent and 1 μg AON were added. Transfection of iPSC and NPC-mn was performed in 12-well plates, mn were transfected in μ-dishes. The two reaction tubes were mixed together, homogenized and incubated for 10 min at room temperature. Afterwards, this transfection mix was distributed dropwise onto the cells. After 24 h of incubation with the transfection mix, the medium was changed as usual.

### Immunocytochemistry

Cells were fixed using 4% paraformaldehyde (PFA) with 0.1% sucrose. Permeabilization of cells was performed with 0.2% Triton in PBS+CaCl₂)/+MgCl₂ for 10 min at room temperature. Unspecific binding was blocked with blocking solution (5% FBS, 10% goat serum in PBS+CaCl₂)/+MgCl₂) for 1 h. Afterwards, the primary antibody was diluted in blocking solution and incubated overnight at 4° C. The antibody solution was removed and after three washing steps for 5 min with PBS+CaCl₂)/+MgCl₂, the Alexa-conjugated secondary antibody was applied for 1 h. Procedure was finished by four washing steps with PBS+CaCl₂)/+MgCl₂ (1 min, 5 min, 5 min, 20 min), one with PBS-CaCl₂)/—MgCl₂ (5 min) and a short time in Millipore water (30 sec). Those washing steps were followed by the removal of the liquid and mounting with ProLong® Gold Antifade Mountant with DAPI which had to dry for at least 4 h in the dark before microscoping.

### Western Blot

Lysis was with RIPA buffer (50 mM Tris, 150 mM NaCl, 0.1% SDS, 1 mM sodium-orthovanadate, 1% NP-40, protease and phosphatase inhibitor). The cells were detached and translocated to a reaction tube and lysed on ice for 15 min. Then, they were homogenized mechanically using a 1 ml syringe with a hollow needle (20 μm diameter) ten times and incubated for further 15 min. After incubation, samples were sonicated 10 times and centrifuged for ten min at 13000 rpm and 4° C. The supernatant containing the protein was transferred to a new reaction tube and the pellet containing the cell debris discarded. With the generated lysates, Bradford Assay was performed and protein concentration calculated to determine volumes for 10 μg protein concentration. Samples were diluted with 4× loading buffer and water and western blot was performed. Proteins were separated by 8% SDS-page and transferred to a polyvinylidene fluoride membrane using the Trans-Blot® turbo transfer system. The membrane was blocked in blocking solution (5% BSA, 0.1% Tween-20) for 1-3 h. Afterwards, the primary antibody was put overnight at 4° C. on the shaker. After washing with 0.2% TBS-T the horse reddish peroxidase conjugated secondary antibody was incubated for 1 h at RT and the residual antibody was again removed by washing with TBS-T. Images were taken using the GelCapture software and analyzed using the GelAnalyzer Software.

### Data Analysis and Statistics

Statistical analyses and graphical representation were performed with GraphPad Prism 6. First, data of all groups was tested for normal distribution using the Shapiro-Wilk test and for homogeneity of variances using the Levene test. Since more than two groups were compared, the analysis of variance (ANOVA) was performed for data that was normally distributed and presented homogeneity of variance to detect whether there are significant differences between their means. If the ANOVA found significant differences, the Tukey post hoc-test was used to determine between which groups these occurred. For data that was distributed non-normally, the Kruskal-Wallis test followed by a post-hoc test was performed for the detection of significant differences between the means of the groups.

Data was shown in bars which represent the means of each group and the standard error of the mean (SEM). Statistical significances were represented by asterisks according to their calculated probability (p-value). For p<0.05, data was considered statistically significant which was indicated with one asterisk (*), for p<0.01 the significant difference was represented by two asterisks (**) and for p<0.001 it was considered statistically highly significant which was represented by three asterisks (***) above the bars.

### Example 4: 50 bp Antisense Oligonucleotides (AONs)

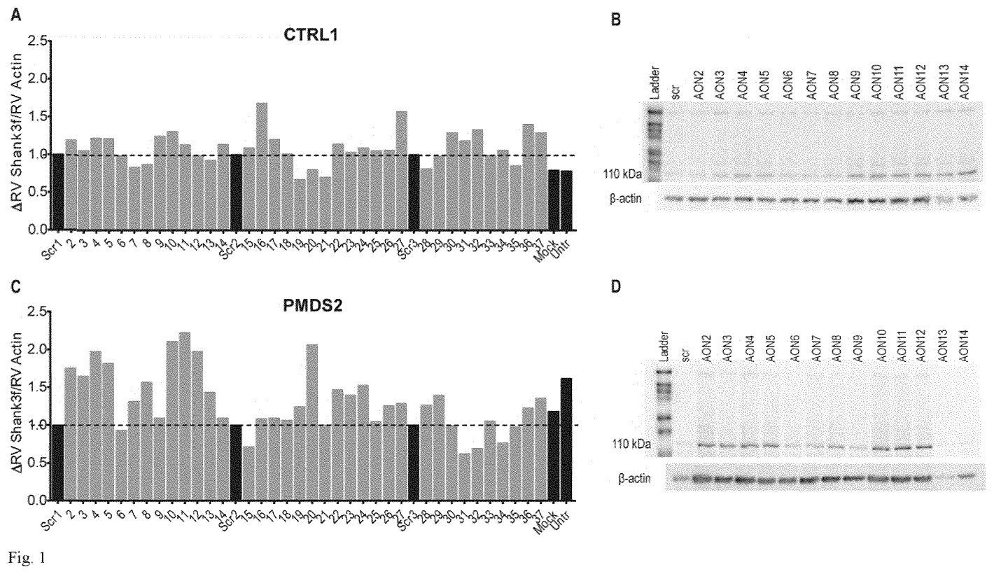

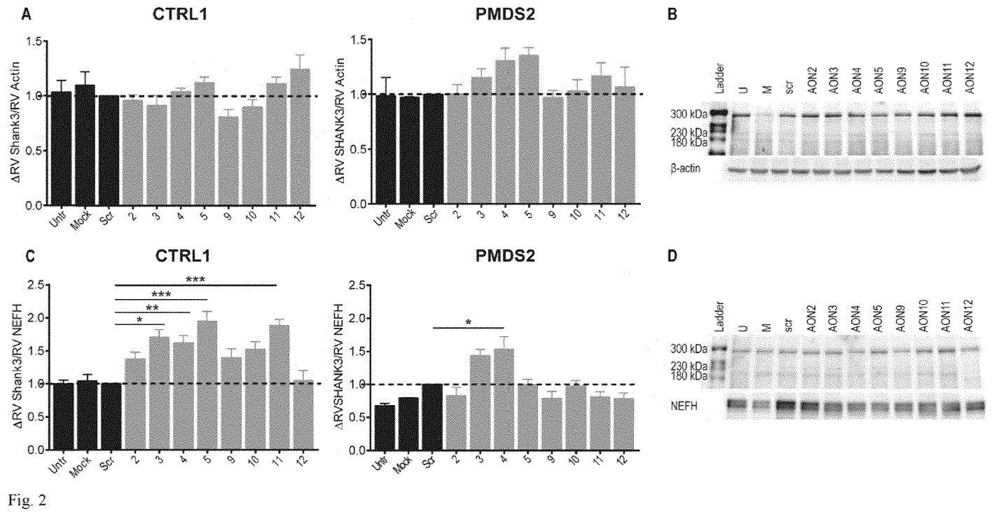

50 bpAONs were generated analog to the human 3′ end of the SHANK3 gene (Table 2). The scrambled AON was generated according to the proportions of the bases in the 50 bp AONs and the sequence of the bases was randomized. AONI could not be generated. All other AONs were used for the screening approach in iPSC. Results of the screening are shown in FIGS. 1 and 2.

### Example 5: 18 bp Antisense Oligonucleotides

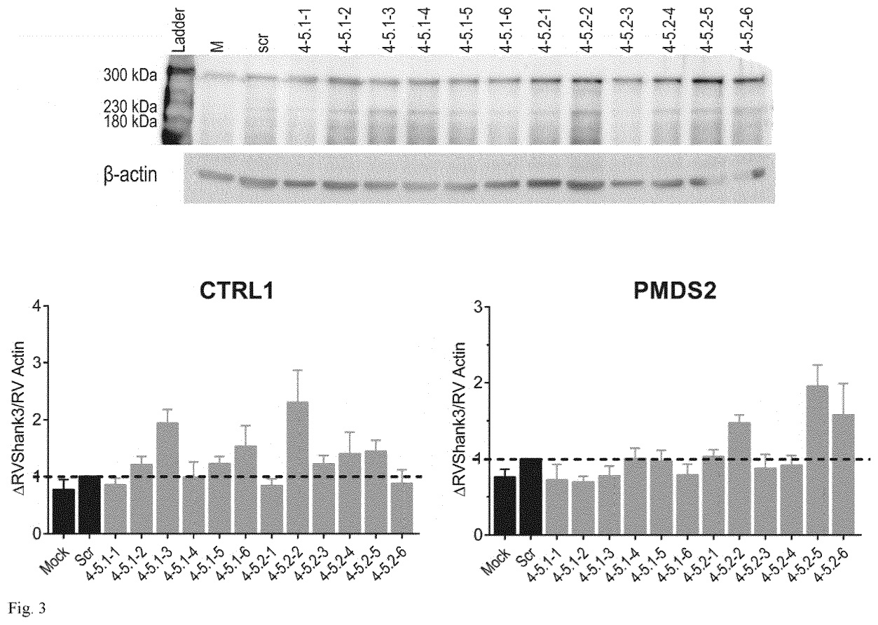

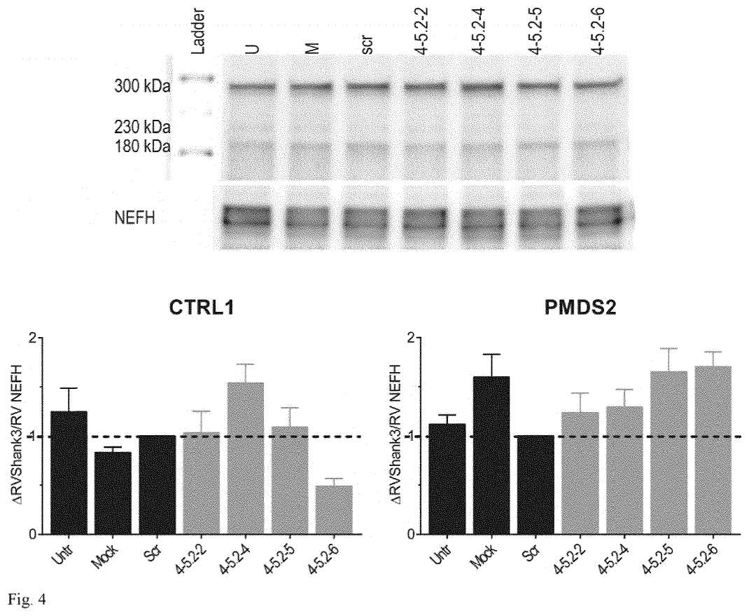

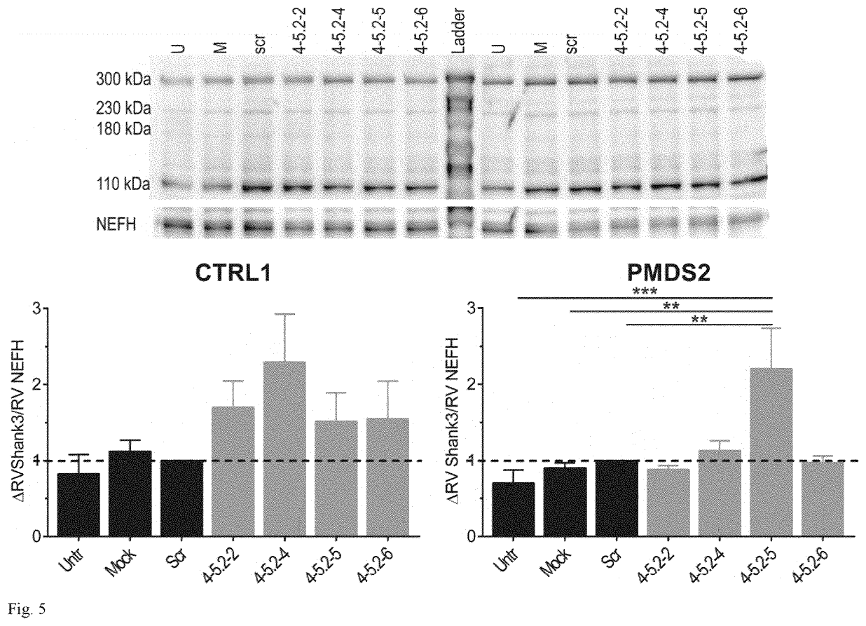

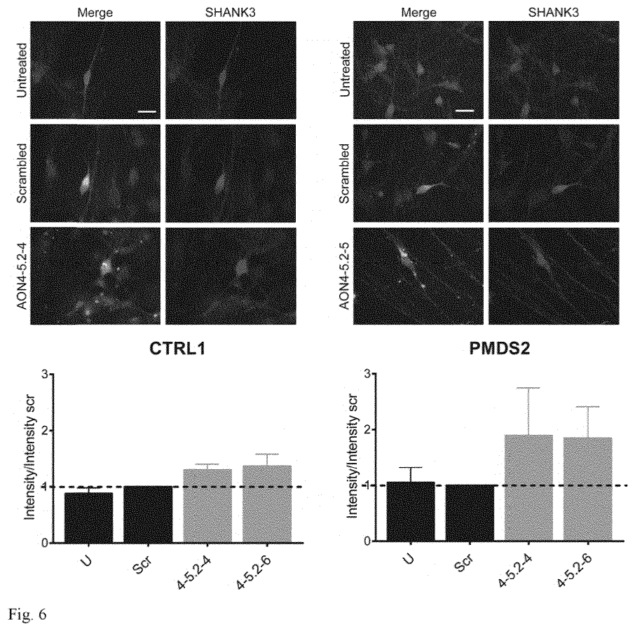

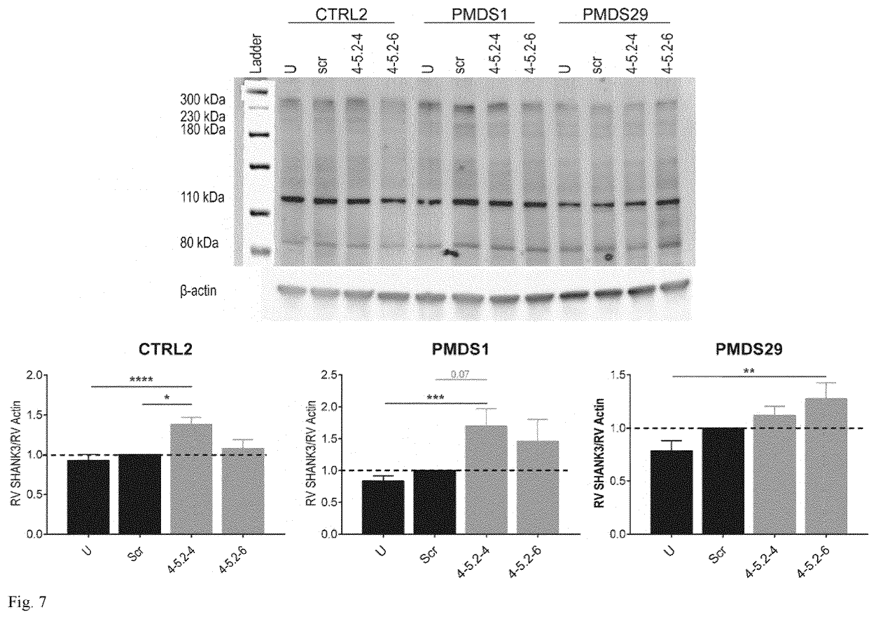

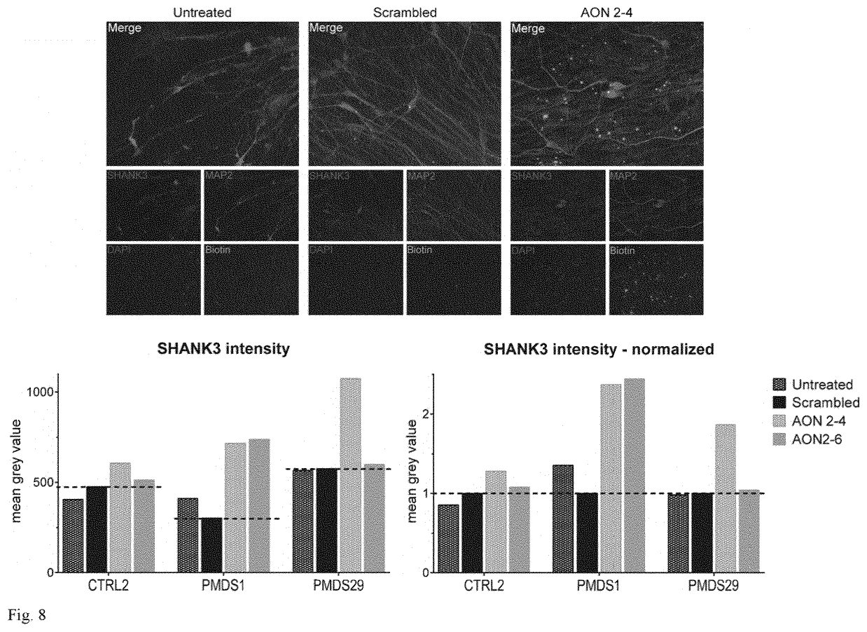

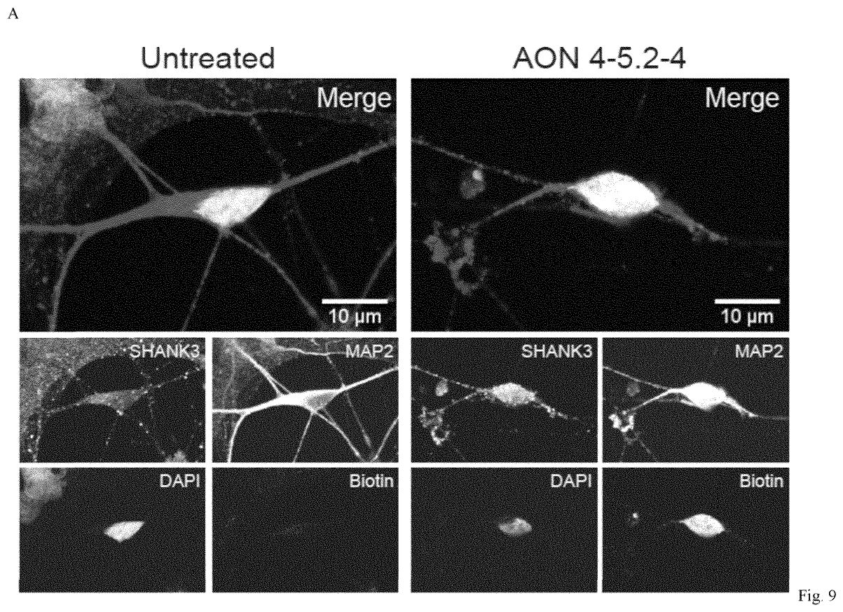

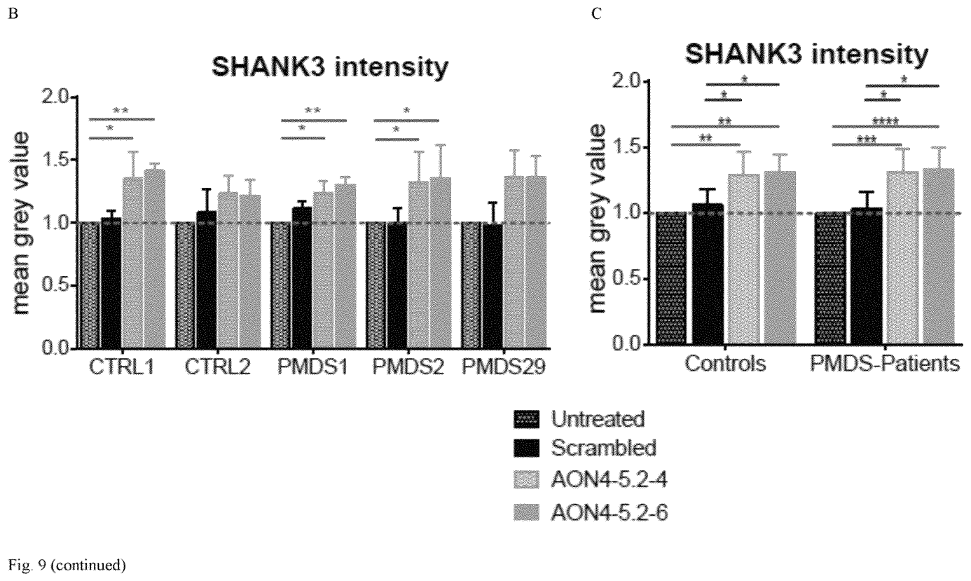

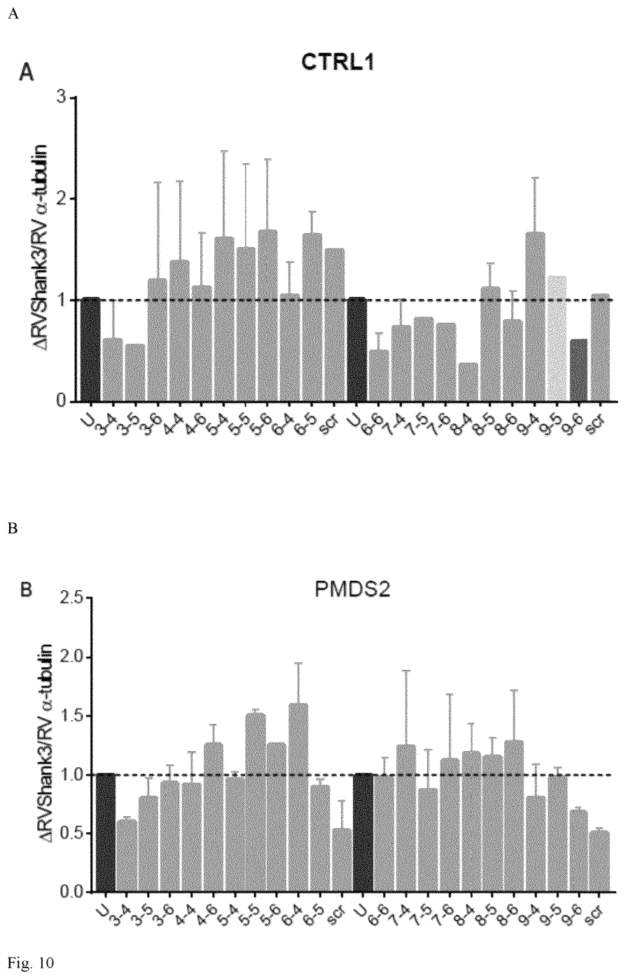

The most effective 50 bp AONs were divided into 18 bp AONs including either the end of the previous AON or the beginning of the next one (Table 3). The scrambled AON was generated according to the proportions of the bases in the 50 bp AONs and the sequence of the bases was randomized. * indicate the location of a PTO backbone and each AON is tagged with a Biotin at the beginning and the end. Results are shown in FIGS. 3 to 8, 9, and 10; clearly, the 18 bp AONs increase the amount of Shank3 protein present in cells transfected therewith.

TABLE 2
50 bp AONs

| | | SEQ |
|---|---|---|
| name | sequence | ID NO |
| scr | GGCGCTCCGATGGTTCCAATGAACACGTCGCCCACGCCGGATGAGCTTCT | 18 |
| AON1 | GGGTGGGGGGCCCTGCCGGCAGGGCACGGCCGGGGCGGGAGTGGGGGTGG | 19 |
| AON2 | TGGCCCGGGCGCCGTCGTAAGGGGCAGGCCGAGCCCGCGGCCCGGGGTGG | 4 |
| AON3 | CAGGGCGAGTCTCCGAGCAACAGCAAACAGGACGATTCATGCAACATTCC | 5 |
| AON4 | GACTGTGCGCTGGGTGGGCTGGGCGGCCGGTGAGGGCACTAAGCAATGTA | 6 |
| AON5 | CCCCACCCCCGGCACCTCCCGCCCCAGCCTCCCTGGTCCACGCCCTTCCT | 7 |
| AON6 | CTGGGAGAATGGCCACCAGGAGCTGCGAAGGAGGTGGTCACGCTAGGGCA | 20 |
| AON7 | CGAGTCACCAAGGCGGGTCCTGACCTCGCATGCTGGACTAGGTTCCCCCT | 21 |
| AON8 | GGGGGGCTCCTTGGCCCCCATCGAGAATCCCAATGTCTCCCCCCTCCCCC | 22 |
| AON9 | TTGTTCCTTTTCTCGTTCCAAATATAGAGTGGATTAAAATATGCAAAACA | 8 |
| AON10 | CCAGTGGAAGTAGAAGGGAGGGGAGAGGAAACTATTACGGACAGAGATAT | 9 |
| AON11 | GCAGTGGCAGTGAGGGGCCGGAGATGGAAGACTGGGCAGCTGCAGTGGGA | 10 |
| AON12 | CCTGGCCAACCCCAGACCAGCTGGAGCGTCCCCTGCCCCGTTGTGGGGTG | 11 |
| AON13 | CTCAGCGAGGCGAGGGGCCGAGCTGGGGCCCCAGGGCGGGCCACTAGGGC | 23 |
| AON14 | GCCCCTCCCACCACGAGCAGCACCTGAAGGGTCGGTGGGGCACACTAGAG | 24 |
| AON15 | CTCCCAGGCCGGCTGGTCCCGCGGGTGCACAGCAGGACCCGCGGCCTGCC | 25 |
| AON16 | GGCTGCGGTGGAGCGGCAGGGCCCTGCCCTCATCCCCCCCGGCCGATGGT | 26 |
| AON17 | ATGGGGACCAAGCTAGCCCCCTCCTTGGGGAGAGACCCTGTGAGGAAGAT | 27 |
| AON18 | ACTTTGGGGTCCAAGGCTGGGAGGCTTCTCTGCTGTCGTTGCCCAAGAGC | 28 |
| AON19 | TGCTCAGACGCACCCAGTTTCACCCACACACATAAAGGGCAGGACAGGAG | 29 |
| AON20 | AGCGGCCCCTGGTGGCCACTCAGTAATCAGGCACACACACGGCTCCCACG | 30 |
| AON21 | ACCCCACGCATGCACACGGTGCACGCCTCCACGGCCCCGCGCTAGTCCAG | 31 |
| AON22 | ATGGCCTCTGACTTTCTTTGGAAGAGGAGACAGGGTGCTCTCACAGGTAC | 32 |
| AON23 | TGAGGCTCTGGAAGGACAGGCTGGGCAAACAGCTGGACCCAGGGTGCAGG | 33 |
| AON24 | GGGCCTCCCCAAGCAGCAGGGATTCACCAGGGAACCCCGCTCAGGCTGGG | 34 |
| AON25 | TGATGCCCTCAGAAGCCCAAGGTGGGGGCGCTGCCTCCAAGGGGCCCTTG | 35 |
| AON26 | CTTTGACTCTCCCCAGGGCCTGTGGTGAACTGACTCTAGGGGTCCCCCTA | 36 |
| AON27 | TCGCTGAGTGTGAGGAGTCACAGTGTGGGGGGCTGGGCACCCTCGGGGGT | 37 |
| AON28 | TCCAAACCCTAGGTTTAAAAACGTCCCAGGGCCCCCCACCCCACAGGTCA | 38 |
| AON29 | GAAATATAAACAGAAACTTGTGAGTGACGTGGATGGAGCTTAGTCCAGAC | 39 |
| AON30 | TTGTGAAAACTTCTGTTTTCTTTTTTTTTTTATTTTATTAAAAAAAGCTA | 40 |
| AON31 | TGGGCCAGGGCGGGGCGGGCAGGCACAGACCGGCGTGCCAGGCCCCTGGG | 41 |
| AON32 | TTGGCGGAAGGATGAGTGGGTGTGACTCTGTGCCCAGGGAGTGGGGCCGG | 42 |
| AON33 | TGTGTATGTGGCAGTCTGTTACAGTGACTGCTGCTGTGTGACCTGGACTG | 43 |
| AON34 | AAAACCCAAATTGAACGGAACCAAAACCCACAGGTGAGTGTGAGACCGAG | 44 |
| AON35 | ATGGAACTCCATACAAAAGGAGGTGAAGCGGAACTGACCCTGTAAAGTTA | 45 |
| AON36 | CGTCAGGGTCAGGAGGCCTCAGGACTGGAGCAGGGGGTGAAACCCCCCGG | 46 |
| AON37 | ATCAATAATAATAATATAAGAAACATAGATCTCTGTGGGGCGTATCACAA | 47 |

TABLE 3
18 bp AONs; * indicates phosphorothioate
nuceotides.

| | | SEQ |
|---|---|---|
| | | ID |
| name | sequence | NO |
| 4-5.1-1 | C*CCCACCCC*CGGCACCT*C | 48 |
| 4-5.1-2 | C*CGCCCCAG*CCTCCCTG*G | 49 |
| 4-5.1-3 | T*CCACGCCC*TTCCTGAC*T | 12 |
| 4-5.1-4 | G*TGCGCTGG*GTGGGCTG*G | 50 |
| 4-5.1-5 | G*CGGCCGGT*GAGGGCAC*T | 51 |
| 4-5.1-6 | A*AGCAATGT*ACAGGGCG*A | 13 |
| 4-5.2-1 | C*TAGGGCAC*CCCACCCC*C | 52 |
| 4-5.2-2 | G*GCACCTCC*CGCCCCAG*C | 14 |
| 4-5.2-3 | C*TCCCTGGT*CCACGCCC*T | 53 |
| 4-5.2-4 | T*CCTGACTG*TGCGCTGG*G | 15 |
| 4-5.2-5 | T*GGGCTGGG*CGGCCGGT*G | 16 |
| 4-5.2-6 | A*GGGCACTA*AGCAATGT*A | 17 |

TABLE 4
additional 18 bp AONs generated from AONs 4 and
5 by shifting the sequence by 2 bases,
when necessary parts from 50 bp long
AON3 or AON6 were included., * indicates
phosphorothioate nuceotides;

| name | sequence | SEQ ID NO: |
|---|---|---|
| 4-5.3-1 | A*GGGCACCC*CACCCCCG*G | 54 |
| 4-5.3-2 | C*ACCTCCCG*CCCCAGCC*T | 55 |
| 4-5.3-3 | C*CCTGGTCC*ACGCCCTT*C | 56 |
| 4-5.3-4 | C*TGACTGTG*CGCTGGGT*G | 57 |
| 4-5.3-5 | G*GCTGGGCG*GCCGGTGA*G | 58 |
| 4-5.3-6 | G*GCACTAAG*CAATGTAC*A | 59 |
| 4-5.4-1 | G*GCACCCCA*CCCCCGGC*A | 60 |
| 4-5.4-2 | C*CTCCCGCC*CCAGCCTC*C | 61 |
| 4-5.4-3 | C*TGGTCCAC*GCCCTTCC*T | 62 |
| 4-5.4-4 | G*ACTGTGCG*CTGGGTGG*G | 63 |
| 4-5.4-5 | C*TGGGCGGC*CGGTGAGG*G | 64 |
| 4-5.4-6 | C*ACTAAGCA*ATGTACAG*G | 65 |
| 4-5.5-1 | C*ACCCCACC*CCCGGCAC*C | 66 |
| 4-5.5-2 | T*CCCGCCCC*AGCCTCCC*T | 67 |
| 4-5.5-3 | G*GTCCACGC*CCTTCCTG*A | 68 |
| 4-5.5-4 | C*TGTGCGCT*GGGTGGGC*T | 69 |
| 4-5.5-5 | G*GGCGGCCG*GTGAGGGC*A | 70 |
| 4-5.5-6 | C*TAAGCAAT*GTACAGGG*C | 71 |
| 4-5.6-1 | C*CACCCCCG*GCACCTCC*C | 72 |
| 4-5.6-2 | G*CCCCAGCC*TCCCTGGT*C | 73 |
| 4-5.6-3 | C*ACGCCCTT*CCTGACTG*T | 74 |
| 4-5.6-4 | G*CGCTGGGT*GGGCTGGG*C | 75 |
| 4-5.6-5 | G*GCCGGTGA*GGGCACTA*A | 76 |
| 4-5.6-6 | G*CAATGTAC*AGGGCGAG*T | 77 |
| 4-5.7-1 | A*CCCCCGGC*ACCTCCCG*C | 78 |
| 4-5.7-2 | C*CCAGCCTC*CCTGGTCC*A | 79 |
| 4-5.7-3 | C*GCCCTTCC*TGACTGTG*C | 80 |
| 4-5.7-4 | G*CTGGGTGG*GCTGGGCG*G | 81 |
| 4-5.7-5 | C*CGGTGAGG*GCACTAAG*C | 82 |
| 4-5.7-6 | A*ATGTACAG*GGCGAGTC*T | 83 |
| 4-5.8-1 | C*CCCGGCAC*CTCCCGCC*C | 84 |
| 4-5.8-2 | C*AGCCTCCC*TGGTCCAC*G | 85 |
| 4-5.8-3 | C*CCTTCCTG*ACTGTGCG*C | 86 |
| 4-5.8-4 | T*GGGTGGGC*TGGGCGGC*C | 87 |
| 4-5.8-5 | G*GTGAGGGC*ACTAAGCA*A | 88 |
| 4-5.8-6 | T*GTACAGGG*CGAGTCTC*C | 89 |
| 4-5.9-1 | C*CGGCACCT*CCCGCCCC*A | 90 |
| 4-5.9-2 | G*CCTCCCTG*GTCCACGC*C | 91 |
| 4-5.9-3 | C*TTCCTGAC*TGTGCGCT*G | 92 |
| 4-5.9-4 | G*GTGGGCTG*GGCGGCCG*G | 93 |
| 4-5.9-5 | T*GAGGGCAC*TAAGCAAT*G | 94 |
| 4-5.9-6 | T*ACAGGGCG*AGTCTCCG*A | 95 |

### LITERATURE CITED

Catanese et al. (2019), Autophagy 15(10):1719-1737
Choi et al. (2015), Mol. Brain 8, 1-12
Darville et al. (2016), EbioMedicine. 9:293-305
DE 1998 122954
Evers et al. (2015), Adv. Drug Deliv. Rev. 87:90-103
Hu & Zhang (2009), Nature Protocols 4(9): 1295-304
Linta et al. (2012), Stem Cells and Development, 21(6):965
Mei et al. (2016), Nature, 530: 481
Phelan and McDermott (2011), Mol. Syndrome!. 2:186-201
Reinhardt et al. (2013), PLOS One 8(11):10.1371/annotation/6a917a2e-df4a-4ad9-99bb-6aa7218b833e
Roussignol et al. (2005), J Neurosci 25(14):3560
Scoles et al. (2017), Nature, 544(7650):362
U.S. Pat. No. 10,131,910
Wang et al. (2019), Mol Psychiatry; doi.org/10.1038/s41380-018-0324-x
WO 2003/025144 A2
WO 2015/190922 A1

1. A method for treatment and/or prevention of Shank3 deficiency in a subject, said method comprising contacting said subject with a polynucleotide comprising a nucleic acid sequence of SEQ ID NO: 1, wherein said oligonucleotide comprises deoxynucleotides and wherein said oligonucleotide comprises a stretch of at least 10 continuous bases essentially complementary to SEQ ID NO:1.

2. The method of claim 1, wherein said Shank3 deficiency is Phelan-McDermid syndrome.

3. The method of claim 1, wherein said oligonucleotide comprises a stretch of from 10 to 100 continuous bases essentially complementary to SEQ ID NO:1.

4. The method of claim 1, wherein said oligonucleotide comprises a stretch of from 10 to 100 continuous bases essentially identical to SEQ ID NO:2.

5. The method of claim 1, wherein said oligonucleotide consists of from 10 to 100 nucleotides.

6. The method of claim 1, wherein said oligonucleotide comprises the nucleic acid sequence of any one of SEQ ID NO:4 to 17.

7. The method of claim 1, wherein said oligonucleotide comprises the nucleic acid sequence of SEQ ID NO:15 or 17.

8. The method of claim 1, wherein said oligonucleotide is a DNA oligonucleotide.

9. The method of claim 1, wherein said oligonucleotide is comprised in a pharmaceutical composition.

10. An oligonucleotide specifically binding to a polynucleotide comprising the nucleic acid sequence of SEQ ID NO:1, wherein said oligonucleotide comprises deoxynucleotides and wherein said oligonucleotide comprises the nucleic acid sequence of any one of SEQ ID NO:4 to 13 or 15 to 17, or consists of the nucleic acid sequence of SEQ ID NO: 14.

11. (canceled)

12. The method of claim 1, comprising contacting said subject with an expression construct encoding said oligonucleotide.

13. The method of claim 1, comprising contacting said subject with a host cell comprising said oligonucleotide.

14. A method for determining whether a subject suffering from Shank3 deficiency is amenable to a therapy with an oligonucleotide as specified in claim 1, comprising
a) contacting a cell sample of said subject with said oligonucleotide;
b) determining the amount of Shank3 polypeptide in said cell sample;
c) comparing the amount of Shank3 polypeptide determined in step b) to a reference; and
d) determining whether a subject suffering from Shank deficiency is amenable to a therapy based on the result of comparison step c).

15. The method of claim 14, wherein said reference is determined based on
(i) a control cell sample of said subject not contacted to said oligonucleotide;
(ii) a population of apparently healthy subjects
(iii) a population of subjects known not to suffer from Shank3 deficiency;
(iv) a population of subjects known to suffer from Shank3 deficiency.

16. (canceled)

17. (canceled)

18. The method of claim 1, wherein said oligonucleotide comprises a stretch of at least 10 continuous bases essentially complementary to nucleotides 1 to 1000 of SEQ ID NO:1.

19. The method of claim 1, wherein said oligonucleotide comprises a stretch of at least 10 continuous bases essentially complementary to nucleotides 50 to 600 of SEQ ID NO:1.

20. The method of claim 1, wherein said oligonucleotide comprises a stretch of at least 10 continuous bases essentially complementary to nucleotides 100 to 300 of SEQ ID NO:1.

21. The method of claim 1, wherein said oligonucleotide comprises a stretch of from 10 to 100 continuous bases essentially identical to SEQ ID NO:3.
